(19) United States
(12) Patent Application Publication
(10) Pub. No.: US 2025/0072951 A1
(43) Pub. Date: Mar. 6, 2025

(54) DEVICE FOR THERAPEUTIC SINO-NASAL TREATMENT

(71) Applicant: Neurent Medical Limited, Oranmore (IE)

(72) Inventors: Cathal McLaughlin, Galway (IE); Steven Horan, Galway (IE); Tom Byrne, Oranmore (IE); Sofia Iniguez De Gante, Galway (IE); Aidan Loughrey, Galway (IE)

(21) Appl. No.: 18/816,456

(22) Filed: Aug. 27, 2024

Related U.S. Application Data
(60) Provisional application No. 63/553,094, filed on Feb. 13, 2024.
(60) Provisional application No. 63/534,960, filed on Aug. 28, 2023.

(51) Int. Cl. A61B 18/14; A61B 18/00

(52) U.S. Cl. CPC A61B 18/14; A61B 2018/0016; A61B 2018/00214; A61B 2018/00577; A61B 2018/1407

(57) ABSTRACT

The invention generally relates to systems and methods for targeting of specific tissue(s) of interest in a sino-nasal region of a patient for the treatment of a rhinosinusitis condition. A device of the present invention includes an end effector comprised of one or more segments, each segment comprising one or more struts which can be in the form of loop-shaped struts or independent struts and each comprising a flexible printed circuit board (PCB) member for delivering energy to one or more target sites within the sino-nasal cavity of the patient while minimizing or avoiding collateral damage to surrounding or adjacent non-targeted tissue, such as blood vessels, bone, and non-targeted neural tissue.

### CROSS-REFERENCE TO RELATED APPLICATIONS

This application claims the benefit of, and priority to, U.S. Provisional Patent Application No. 63/534,960, filed Aug. 28, 2023, and U.S. Provisional Patent Application No. 63/553,094, filed Feb. 13, 2024, the content of each of which is incorporated by reference herein in its entirety.

### FIELD OF THE INVENTION

The invention generally relates to systems and methods for treating medical conditions, and, more particularly, to therapeutically modulating nerves in a nasal region of a patient for the treatment of a rhinosinusitis condition.

### BACKGROUND

Rhinitis is an inflammatory disease of the nose and is reported to affect up to 40% of the population. It is the fifth most common chronic disease in the United States. The most common and impactful symptoms of rhinitis are congestion and rhinorrhea. Allergic rhinitis accounts for up to 65% of all rhinitis patients. Allergic rhinitis is an immune response to an exposure to allergens, such as airborne plant pollens, pet dander or dust. Non-allergic rhinitis is the occurrence of common rhinitis symptoms of congestion and rhinorrhea. As non-allergic rhinitis is not an immune response, its symptoms are not normally seasonal and are often more persistent. The symptoms of rhinitis include a runny nose, sneezing, and sino-nasal itching and congestion.

Current treatment devices generally provide therapeutic treatment using various modalities, such as cryotherapeutic cooling, ultrasound energy (e.g., high intensity focused ultrasound (“HIFU”) energy), microwave energy (e.g., via a microwave antenna), direct heating, high and/or low power laser energy, mechanical vibration, and/or optical power. Delivery of such therapeutic treatment is provided via one or more discrete elements (e.g., electrodes, transducers, etc.) provided on some form of an end effector.

### SUMMARY

The invention recognizes that a problem with current surgical procedures is that such procedures are not accurate and cause significant collateral damage in order to treat rhinitis. The invention is directed to new and unique end effector designs to provide an overall improved medical device for the treatment of a rhinosinusitis condition. In particular, end effector includes one or more retractable and expandable segments or stages, each of which is comprised of a framework of support elements, generally in the form of struts, having elastic properties.

Each retractable and expandable segment may further include one or more flexible printed circuit board (PCB) members provided thereon. The flexible PCB members are composed of a flexible material capable of moving (e.g., bending, twisting, folding, etc.) between various positions in correspondence with movement of the underlying retractable and expandable segment to which it is attached. Each flexible PCB member further includes one or more energy delivering elements (e.g., electrodes) provided thereon and configured to deliver energy to tissue associated with one or more target sites in the sino-nasal cavity. Once delivered within the sino-nasal cavity, the one or more segments can expand to a specific shape and/or size corresponding to anatomical structures within the sino-nasal cavity and associated with target sites to undergo delivery of therapeutic energy for treatment of a condition (i.e., rhinosinusitis or the like). As such, once deployed, the flexible PCBs of the one or more segments contact and conform to a shape of the respective locations, including conforming to and complementing shapes of the one or more anatomical structures, thereby accurately positioning the electrodes for focused application of energy to targeted tissue at the one or more target sites.

The present invention utilizes the many benefits of flexible PCBs, as well as certain manufacturing techniques, to provide an end effector that is capable of highly conforming to anatomical variations within a sino-nasal cavity so that an operator can perform an accurate, minimally invasive, and localized application of energy to one or more target sites within the sino-nasal cavity of the patient to thereby treat a sino-nasal condition.

In particular, the underlying design of the end effector is unique. For example, in one embodiment, the end effector includes a single segment having a bilateral geometry. In particular, the single elements includes two identical sides, including a first side formed of struts and a second side formed of struts. This bilateral geometry allows at least one of the two sides to conform to and accommodate an anatomical structure within the sino-nasal cavity when the segment in an expanded state. For example, when in the expanded state, the plurality of struts contact multiple locations along multiple portions of the anatomical structure and electrodes provided by the struts are configured to emit energy at a level sufficient to create multiple micro-lesions in tissue of the anatomical structure that interrupt neural signals to mucus producing and/or mucosal engorgement elements.

For example, the bilateral geometry refers to the fact that segments of one side of the end effector accommodate various anatomical locations along the lateral wall of one nasal cavity, while segments on the opposing side of the end effector accommodate locations on the lateral wall within the other nasal cavity. It should be noted that, in other embodiments, the end effector is multi-segmented in that it is composed of two or more segments, each having a bilateral geometry as previously described. Furthermore, in other embodiments, the end effector may include a single segment or multiple segments, each having a unilateral geometry.

Furthermore, each segment of the end effector may be formed as a unitary work piece having elastic properties. More specifically, a single piece of shape memory material, such as nitinol, may be used to construct one or more portions of a given segment. The single workpiece may initially be in the form of a tube or a flat plate and can be laser cut to form the desired framework of support elements of the proximal and distal segments. In addition to reducing time, cost, and complexity, the use of laser machining allows a greater amount of design freedom for the manufacturer, in turn leading to a more tailored geometry and mechanical properties of a given segment of the end effector. For example, laser machining allows greater control over mechanical properties of the support elements, including tailoring the stiffness of a specific one of, or a given group of, support elements for a given segment, thereby allowing for tailoring of the tissue apposition profile when the given segment is in an expanded, deployed configuration.

Furthermore, utilizing a stock workpiece in the form of a tube or flat plate results in support elements having a relatively flat surface upon which a corresponding flexible PCB member is affixed, thereby improving apposition of the PCB member to tissue within the sino-nasal cavity.

Furthermore, the use of flexible PCB members results in a greater amount of usable surface area than what is otherwise available with existing end effectors. The increase in surface area allows for a greater number of energy delivery elements to be introduced and utilized in a given procedure and further expands the possible number of patterns of such energy delivery elements. As a result, the contact surface increases substantially, thereby allowing for the end effector of the present invention to deliver treatment to certain areas within the sino-nasal cavity that may have been previously unreachable or untreatable with current treatment devices, or that previously required a surgeon to reposition a given device to reach such areas. The use of flexible PCB members also reduces the overall complexity with regard to manufacturing the end effector of the present invention. In particular, any given flexible PCB member (including an overall PCB assembly, which includes multiple PCB members) is constructed separately from the end effector, which includes constructing the overall electrode design and placement on a given PCB member. Once a PCB assembly is complete, PCB members are then attached to respective portions of a given segment of the end effector as a separate manufacturing step, thereby reducing the complexity that is otherwise associated with placing electrodes directly on the end effector, which is a common practice. In some embodiments, one or more of the support elements of an underlying segment of the end effector may further include fixation points that facilitate the attachment and alignment of polymer sleeves and/or flexible PCB members thereto.

The present invention further provides unique mechanisms for deploying the end effector (i.e., transitioning a given segment from between a retracted configuration and an expanded, deployed configuration). In one embodiment, a treatment device includes a mechanical expansion assembly, which is generally in the form of a sphere or the like, that coupled to an input mechanism of a handle of the device via a pull rod or wire. In particular, the sphere is positioned at a distal-most end of an end effector, specifically positioned so as to be drawn towards a center of a segment and thereby cause support elements or struts of the given segment to expand and transition to the deployed configuration upon user input with the pull rod or wire. More specifically, a user need only pull back on the pull rod or wire, which in turn draws the sphere towards the handle, thereby forcing the support elements or struts of the segment to extend outwardly and in a radial direction so as to expand.

In another embodiment, the mechanical expansion assembly includes a push/pull rod assembly. For example, in one embodiment, a segment may include multiple struts or support elements that are connected to one another at a distal-most end, such as a cap or the like. A push/pull rod may have a distal-most end coupled to the cap and a proximal-most end associated with an input mechanism of a handle of the device. Accordingly, a user need only manipulate the input mechanism to either cause the push/pull rod to be drawn towards the handle, thereby drawing the cap towards the handle and resulting in the segment expanding in a radial direction and transition to a deployed configuration or cause the push/pull rod to extend away from the handle, thereby pushing the cap away from the handle and resulting in the segment contracting and transitioning to a lower-profile, retracted configuration. In a similar embodiment, the mechanical expansion assembly includes an aperture arm (in the form of a cap or the like) that is coupled to each of the struts or support elements of the segment via respective cross members. The arm is positioned at a distal-most end of a push/pull rod. Accordingly, a user need only manipulate an input mechanism on the handle to either cause the push/pull rod to be drawn towards the handle, thereby drawing the aperture arm towards the handle and resulting in the segment expanding in a radial direction and transition to a deployed configuration or cause the push/pull rod to extend away from the handle, thereby pushing the arm away from the handle and resulting in the segment contracting and transitioning to a lower-profile, retracted configuration. The various mechanical expansion assemblies improve contact between energy delivering elements of the end effector and the target sites within the nasal cavity, thereby improving the overall chances of achieving successful treatment.

Accordingly, the end effector of the present invention addresses drawbacks of current treatment devices. In particular, the invention recognizes that current treatment devices may have limitations regarding the delivery of therapeutic energy to tissues of interest, as the delivery of energy may be limited by the architecture of a given end effector. More specifically, many current devices include an end effector having a number of energy delivering elements discretely placed along portions thereof, which results in a surgeon having to reposition the end effector and deliver energy multiple times to a single target site to ensure that the tissue of interest receives adequate treatment. Accordingly, such devices may result in incomplete treatment of the targeted tissue and/or collateral damage to surrounding tissue, organs, bone, or the like.

### BRIEF DESCRIPTION OF THE DRAWINGS

FIGS. 1A and 1B are diagrammatic illustrations of a system for treating a condition of a patient using a handheld device according to some embodiments of the present disclosure.

FIG. 2 is a diagrammatic illustration of the console coupled to the handheld device consistent with the present disclosure, further illustrating one embodiment of an end effector of the handheld device for delivering energy to tissue at one or more target sites.

FIG. 3A is a cut-away side view illustrating the anatomy of a lateral sino-nasal wall.

FIG. 3B is an enlarged side view of the nerves of the lateral sino-nasal wall of FIG. 3A.

FIG. 3C is a front view of a left palatine bone illustrating geometry of microforamina in the left palatine bone.

FIG. 4 is a side view of one embodiment of a handheld device for providing therapeutic treatment consistent with the present disclosure.

FIGS. 5A and 5B are perspective and front (proximal facing) views, respectively, of one embodiment of a single segment having a bilateral geometry, illustrating a framework of loop-shaped struts of the single segment.

FIG. 5C is a perspective view of another embodiment of a single segment having a bilateral geometry, illustrating a framework of independent struts of the single segment.

FIGS. 6A and 6B are perspective views of embodiments of a single segment that comprises of looped-shaped struts (FIG. 6A) and independent struts (FIG. 6B), respectively, in an expanded, deployed configuration and including a flexible printed circuit board (PCB) assembly thereon.

FIG. 7A is a perspective view illustrating placement of polymer caps or tubing to the free, distal ends of the struts to create atraumatic tips or close a loop and couple two struts to one another, respectively.

FIG. 7B is a perspective exploded view illustrating the interaction and assembly of components with one another in an exemplary embodiment of a single segment having a bilateral geometry.

FIG. 8 is a perspective view illustrating loading of molded polymer tips to free distal ends of independent struts.

FIGS. 9A and 9B are perspective views of other embodiments of a single segment that comprises of looped-shaped struts (FIG. 9A) and independent struts (FIG. 9B), respectively, in an expanded, deployed configuration and including a flexible printed circuit board (PCB) assembly thereon.

FIG. 10 is a side view of one embodiment of a multi-segment end effector composed of two separate segments (first and second segments) having a bilateral geometry, specifically illustrating the unitary construction of the framework of each.

FIGS. 11A and 11B are perspective views of embodiments of a multi-segment end effector in which each segment is comprised of looped-shaped struts (FIG. 11A) and independent struts (FIG. 11B), respectively, in an expanded, deployed configuration and including a flexible printed circuit board (PCB) assembly thereon.

FIG. 12A is a cut-away side view illustrating one approach for delivering a single segment end effector having a bilateral geometry to a target site within a nasal region in accordance with embodiments of the present disclosure.

FIG. 12B provides additional views illustrating an approach for delivering a single segment end effector having a bilateral geometry to a target site within a nasal region in accordance with embodiments of the present disclosure.

FIG. 12C provides another view illustrating an approach for delivering a single segment end effector having a bilateral geometry to a target site within a nasal region in accordance with embodiments of the present disclosure.

FIG. 12D provides additional views illustrating an approach for delivering a single segment end effector having a bilateral geometry to a target site within a nasal region in accordance with embodiments of the present disclosure.

FIG. 13 is a perspective view of one embodiment of a single segment having a unilateral geometry, illustrating a framework of independent struts of the single segment.

FIGS. 14A and 14B are perspective views of embodiments of a single segment having a unilateral geometry that comprises of looped-shaped struts (FIG. 14A) and independent struts (FIG. 14B), respectively, in an expanded, deployed configuration and including a flexible printed circuit board (PCB) assembly thereon.

FIGS. 15A and 15B are side views of one embodiment of a framework of the single segment having a unilateral geometry in which the independent struts have a staggered take off section, resulting in transitioning of each independent strut between a retracted configuration and expanded, deployed configuration in a stepwise manner.

FIG. 16 is a perspective view of an embodiment of a multi-segment end effector composed of two segments that each have a unilateral geometry.

FIG. 17 is a perspective view illustrating attachment of an interstage component to a given segment of the multi-segment end effector.

FIG. 18 is a perspective view illustrating joining of adjacent independent struts to one another via placement of a wire or braided tube over respective distal ends of the adjacent struts.

FIG. 19 is a perspective view of a multi-segment end effector having at least two segments each having a unilateral geometry and comprising looped-shaped struts in an expanded, deployed configuration and including a flexible printed circuit board (PCB) assembly thereon.

FIG. 20 is a cut-away side view illustrating one approach for delivering a single segment end effector having a unilateral geometry to a target site within a nasal region in accordance with embodiments of the present disclosure.

FIGS. 21 and 22 show user interaction with a handle of the treatment device of the present disclosure, including application of therapeutic treatment to target sites via a single segment having a unilateral geometry.

FIGS. 23, 24, 25A, 25B, and 25C are views of one embodiment of a mechanical expansion assembly for deploying an end effector consistent with the present disclosure.

FIG. 23 is a side view of a polymer sphere mechanical expansion assembly in which the segment of the end effector is in a retracted configuration.

FIG. 24 is a perspective view, partly in phantom, illustrating the polymer sphere mechanical expansion assembly and further showing the segment of the end effector in an expanded, deployed configuration.

FIGS. 25A, 25B, and 25C illustrate transitioning of the segment of the end effector from a retracted configuration to an expanded, deployed configuration based on user manipulation of the polymer sphere mechanical expansion assembly.

FIGS. 26A and 26B are perspective views illustrating another embodiment of a mechanical expansion assembly that includes a push/pull rod assembly for transitioning a segment of the end effector between the retracted configuration (FIG. 26A) and the expanded, deployed configuration (FIG. 26B).

FIGS. 27A and 27B are perspective views illustrating another embodiment of a mechanical expansion assembly that includes a push/pull rod assembly for transitioning a segment of the end effector between the retracted configuration (FIG. 27A) and the expanded, deployed configuration (FIG. 27B).

FIG. 28 is a perspective view of one embodiment of a handheld device for providing therapeutic treatment consistent with the present disclosure.

FIG. 29 is an enlarged view of a portion of the single segment end effector of FIG. 28 illustrating a looped-shaped strut consistent with the present disclosure.

FIGS. 30A and 30B are plan views of exemplary electrode arrangements provided on loop-shaped struts. FIG. 30A shows a staggered electrode arrangement and FIG. 30B shows an aligned electrode arrangement.

FIG. 31 is a perspective view of an exemplary user interface provided on a display of a console consistent with the present disclosure.

FIG. 32 is a side view, partly in section, illustrating the anatomy of a sino-nasal cavity specific locations within the sino-nasal cavity that a single segment end effector having a bilateral geometry is positioned for delivery of treatment energy in accordance with embodiments of the present disclosure.

FIGS. 33A and 33B are side sectional views illustrating positioning of the single segment end effector at first and second positions, respectively, within the sino-nasal cavity.

### DETAILED DESCRIPTION

There are various conditions related to the sino-nasal cavity which may impact breathing and other functions of the nose. One of the more common conditions is rhinitis, which is defined as inflammation of the membranes lining the nose. The symptoms of rhinitis include sino-nasal blockage, obstruction, congestion, sino-nasal discharge (e.g., rhinorrhea and/or posterior sino-nasal drip), facial pain, facial pressure, and/or reduction or complete loss of smell and/or taste. Sinusitis is another common condition, which involves an inflammation or swelling of the tissue lining the sinuses. Rhinitis and sinusitis are frequently associated with one another, as sinusitis is often preceded by rhinitis. Accordingly, the term rhinosinusitis is often used to describe both conditions.

Depending on the duration and type of systems, rhinosinusitis can fall within different subtypes, including allergic rhinitis, non-allergic rhinitis, chronic rhinitis, acute rhinitis, recurrent rhinitis, chronic sinusitis, acute sinusitis, recurrent sinusitis, and medical resistant rhinitis and/or sinusitis, in addition to combinations of one or more of the preceding conditions. It should be noted that an acute rhinosinusitis condition is one in which symptoms last for less than twelve weeks, whereas a chronic rhinosinusitis condition refers to symptoms lasting longer than twelve weeks.

A recurrent rhinosinusitis condition refers to four or more episodes of an acute rhinosinusitis condition within a twelve-month period, with resolution of symptoms between each episode. There are numerous environmental and biological causes of rhinosinusitis. Non-allergic rhinosinusitis, for example, can be caused by environmental irritants, medications, foods, hormonal changes, and/or sino-nasal septum deviation. Triggers of allergic rhinitis can include exposure to seasonal allergens, perennial allergens that occur any time of year, and/or occupational allergens. Accordingly, rhinosinusitis affects millions of people and is a leading cause for patients to seek medical care.

The invention recognizes that a problem with current surgical procedures is that such procedures are not accurate, cause significant collateral damage, and are limited in scope of treatment. The invention solves that problem by providing a treatment device having a unique end effector configured to complement anatomy a multiple different locations within the sino-nasal cavity. The end effector includes one or more retractable and expandable segments, each of which is comprises a framework of support elements having clastic properties. Once delivered within the sino-nasal cavity, the one or more segments can expand to a specific shape and/or size corresponding to anatomical structures within the sino-nasal cavity and associated with target sites to undergo delivery of therapeutic energy for treatment of a condition (i.e., rhinosinusitis or the like).

In particular, the underlying design of the end effector is unique. For example, in one embodiment, the end effector includes a single segment having a bilateral geometry. In particular, the single element includes two identical sides, including a first side formed of struts and a second side formed of struts. This bilateral geometry allows at least one of the two sides to conform to and accommodate an anatomical structure within the sino-nasal cavity when the segment in an expanded state. For example, when in the expanded state, the plurality of struts contact multiple locations along multiple portions of the anatomical structure and electrodes provided by the struts are configured to emit energy at a level sufficient to create multiple micro-lesions in tissue of the anatomical structure that interrupt neural signals to mucus producing and/or mucosal engorgement elements.

For example, the bilateral geometry refers to the fact that segments of one side of the end effector accommodate various anatomical locations along the lateral wall of one nasal cavity (i.e., left nasal cavity), while segments on the opposing side of the end effector accommodate locations on the lateral wall within the other nasal cavity (i.e., right nasal cavity). It should be noted that, in other embodiments, the end effector is multi-segmented in that it is composed of two or more segments, each having a bilateral geometry as previously described. Furthermore, in other embodiments, the end effector may include a single segment or multiple segments, each having a unilateral geometry.

The advantage of the single stage bilateral geometry is that it is simpler from a design and cost perspective and can be used to treat multiple locations in the middle meatus and posterior to the middle turbinate along the lateral wall of each nasal cavity.

Furthermore, each segment of the end effector may be formed as a unitary work piece having elastic properties. More specifically, a single piece of shape memory material, such as nitinol, may be used to construct one or more portions of a given segment. The single workpiece may initially be in the form of a tube or a flat plate and can be laser cut to form the desired framework of support elements of the proximal and distal segments. In addition to reducing time, cost, and complexity, the use of laser machining allows a greater amount of design freedom for the manufacturer, in turn leading to a more tailored geometry and mechanical properties of a given segment of the end effector. For example, laser machining allows greater control over mechanical properties of the support elements, including tailoring the stiffness of a specific one of, or a given group of, support elements for a given segment, thereby allowing for tailoring of the tissue apposition profile when the given segment is in an expanded, deployed configuration.

Each retractable and expandable segment comprises one or more flexible printed circuit board (PCB) members provided thereon. The flexible PCB members are composed of a flexible material capable of moving (e.g, bending, twisting, folding, etc.) between various positions in correspondence with movement of the underlying retractable and expandable segment to which it is attached. Each flexible PCB member further includes one or more energy delivering elements (e.g., electrodes) provided thereon and configured to deliver energy to tissue associated with one or more target sites in the sino-nasal cavity.

The present invention utilizes the many benefits of flexible PCBs, as well as certain manufacturing techniques, to provide an end effector that is capable of highly conforming to anatomical variations within a sino-nasal cavity so that an operator can perform an accurate, minimally invasive, and localized application of energy to one or more target sites within the sino-nasal cavity of the patient to thereby treat a sino-nasal condition.

In this manner, the present invention provides an end effector that is capable of highly conforming to anatomical variations within a sino-nasal cavity so that an operator can perform an accurate, minimally invasive, and localized application of energy to one or more target sites within the sino-nasal cavity of the patient to thereby treat a sino-nasal condition.

Accordingly, a handheld device of the present invention provides a user-friendly, non-invasive means of treating rhinosinusitis conditions, including precise and focused application of energy to the intended targeted tissue without causing collateral and unintended damage or disruption to other tissue and/or structures. Thus, the efficacy of a vidian neurectomy procedure can be achieved with the systems and methods of the present invention without the drawbacks discussed above. Most notably, the handheld device provides a surgeon with a user-friendly, non-invasive, and precise means for treating rhinorrhea and other symptoms of rhinosinusitis by targeting only those specific structures associated with such conditions, thereby ensuring that such treatment is effective at treating rhinosinusitis conditions while greatly reducing the risk of causing lateral damage or disruption to other tissue and/or structure thereby reducing the likelihood of unintended complications and side effects.

It should be noted that, although many of the embodiments are described with respect to devices, systems, and methods for therapeutically modulating nerves associated with the peripheral nervous system (PNS) and thus the treatment of peripheral neurological conditions or disorders, other applications and other embodiments in addition to those described herein are within the scope of the present disclosure. For example, at least some embodiments of the present disclosure may be useful for the treatment of other disorders, such as the treatment of disorders associated with the central nervous system.

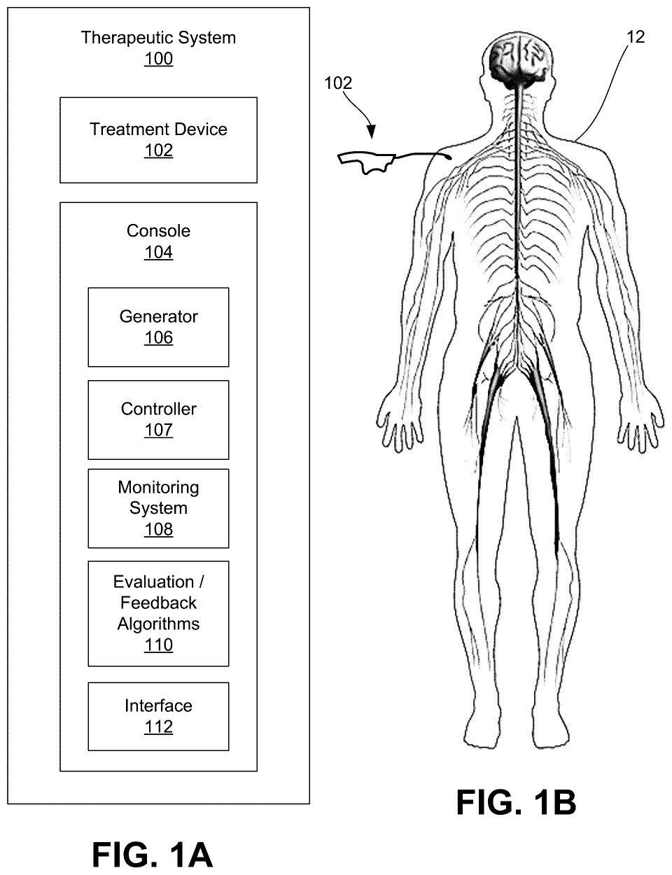

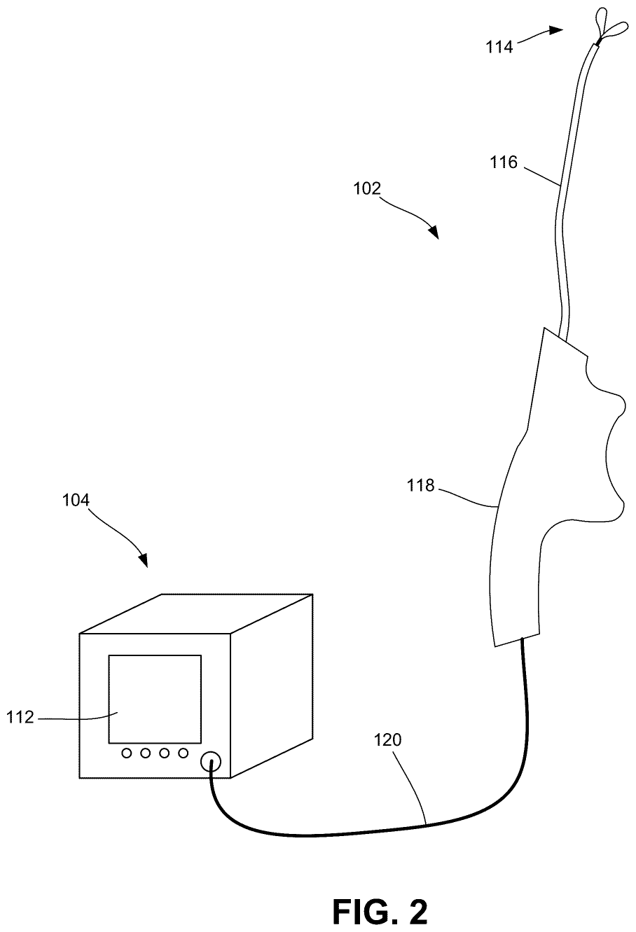

FIGS. 1A and 1B are diagrammatic illustrations of a therapeutic system 100 for treating a condition of a patient using a handheld device 102 according to some embodiments of the present disclosure. The system 100 generally includes a device 102 and a console 104 to which the device 102 is to be connected. FIG. 2 is a diagrammatic illustrations of the console 104 coupled to the handheld device 102 illustrating an exemplary embodiment of an end effector 114 for delivering energy to tissue at the one or more target sites of a patient for the treatment of a condition. As illustrated, the device 102 is a handheld device, which includes end effector 114, a shaft 116 operably associated with the end effector 114, and a handle 118 operably associated with the shaft 116. The end effector 114 may be collapsible/retractable and expandable, thereby allowing for the end effector 114 to be minimally invasive (i.e., in a collapsed or retracted state) upon delivery to one or more target sites within a patient and then expanded once positioned at the target site. It should be noted that the terms “end effector” and “therapeutic assembly” may be used interchangeably throughout this disclosure.

For example, a surgeon or other medical professional performing a procedure can utilize the handle 118 to manipulate and advance the shaft 116 to a desired target site, wherein the shaft 116 is configured to locate at least a distal portion thereof intraluminally at a treatment or target site within a portion of the patient associated with tissue to undergo electrotherapeutic stimulation for subsequent treatment of an associated condition or disorder. In the event that the tissue to be treated is a nerve, such that electrotherapeutic stimulation thereof results in treatment of an associated neurological condition, the target site may generally be associated with peripheral nerve fibers. The target site may be a region, volume, or area in which the target nerves are located and may differ in size and shape depending upon the anatomy of the patient. Once positioned, the end effector 114 may be deployed and subsequently deliver energy to the one or more target sites. The energy delivered may be non-therapeutic stimulating energy at a frequency for locating neural tissue and further sensing one or more properties of the neural tissue. For example, the end effector 114 may include an electrode array, which includes at least a subset of electrodes configured to sense the presence of neural tissue at a respective position of each of the electrodes, as well as morphology of the neural tissue, wherein such data may be used for determining, via the console 104, the type of neural tissue, depth of neural tissue, and location of neural tissue.

Based on the identification of the neural tissue type, the console 104 is configured to determine a specific treatment pattern for controlling delivery of energy from the end effector 114 upon the target site at a specific level for a specific period of time to the tissue of interest (i.e., the targeted tissue) sufficient to ensure successful ablation/modulation of the targeted tissue while minimizing and/or preventing collateral damage to surrounding or adjacent non-targeted tissue at the target site. Accordingly, the end effector 114 is able to therapeutically modulate nerves of interest, particularly nerves associated with a peripheral neurological conditional or disorder so as to treat such condition or disorder, while minimizing and/or preventing collateral damage.

For example, the end effector 114 may include at least one energy delivery element, such as an electrode, configured to delivery energy to the target tissue which may be used for sensing presence and/or specific properties of tissue (such tissue including, but not limited to, muscle, nerves, blood vessels, bones, etc.) for therapeutically modulating tissues of interest, such as neural tissue. For example, one or more electrodes may be provided by one or more portions of the end effector 114, wherein the electrodes may be configured to apply electromagnetic neuromodulation energy (e.g., radiofrequency (RF) energy) to target sites. In other embodiments, the end effector 114 may include other energy delivery elements configured to provide therapeutic neuromodulation using various other modalities, such as cryotherapeutic cooling, ultrasound energy (e.g., high intensity focused ultrasound (“HIFU”) energy), microwave energy (e.g., via a microwave antenna), direct heating, high and/or low power laser energy, mechanical vibration, and/or optical power.

In some embodiments, the end effector 114 may include one or more sensors (not shown), such as one or more temperature sensors (e.g., thermocouples, thermistors, etc.), impedance sensors, and/or other sensors. The sensors and/or the electrodes may be connected to one or more wires extending through the shaft 116 and configured to transmit signals to and from the sensors and/or convey energy to the electrodes.

As shown, the device 102 is operatively coupled to the console 104 via a wired connection, such as cable 120. It should be noted, however, that the device 102 and console 104 may be operatively coupled to one another via a wireless connection. The console 104 is configured to provide various functions for the device 102, which may include, but is not limited to, controlling, monitoring, supplying, and/or otherwise supporting operation of the device 102. For example, when the device 102 is configured for electrode-based, heat-element-based, and/or transducer-based treatment, the console 104 may include an energy generator 106 configured to generate RF energy (e.g., monopolar, bipolar, or multi-polar RF energy), pulsed electrical energy, microwave energy, optical energy, ultrasound energy (e.g., intraluminally-delivered ultrasound and/or HIFU), direct heat energy, radiation (e.g., infrared, visible, and/or gamma radiation), and/or another suitable type of energy.

In some embodiments, the console 104 may include a controller 107 communicatively coupled to the device 102. However, in the embodiments described herein, the controller 107 may generally be carried by and provided within the handle 118 of the device 102. The controller 107 is configured to initiate, terminate, and/or adjust operation of one or more electrodes provided by the end effector 114 directly and/or via the console 104. For example, the controller 107 can be configured to execute an automated control algorithm and/or to receive control instructions from an operator (e.g., surgeon or other medical professional or clinician). For example, the controller 107 and/or other components of the console 104 (e.g., processors, memory, etc.) can include a computer-readable medium carrying instructions, which when executed by the controller 107, causes the device 102 to perform certain functions (e.g., apply energy in a specific manner, detect impedance, detect temperature, detect nerve locations or anatomical structures, etc.). A memory includes one or more of various hardware devices for volatile and non-volatile storage, and can include both read-only and writable memory. For example, a memory can comprise random access memory (RAM), CPU registers, read-only memory (ROM), and writable non-volatile memory, such as flash memory, hard drives, floppy disks, CDs, DVDs, magnetic storage devices, tape drives, device buffers, and so forth. A memory is not a propagating signal divorced from underlying hardware; a memory is thus non-transitory.

The console 104 may further be configured to provide feedback to an operator before, during, and/or after a treatment procedure via evaluation/feedback algorithms 110. For example, the evaluation/feedback algorithms 110 can be configured to provide information associated with the location of nerves at the treatment site, the temperature of the tissue at the treatment site, and/or the effect of the therapeutic neuromodulation on the nerves at the treatment site. In certain embodiments, the evaluation/feedback algorithm 110 can include features to confirm efficacy of the treatment and/or enhance the desired performance of the system 100.

For example, the evaluation/feedback algorithm 110, in conjunction with the controller 107, can be configured to monitor temperature at the treatment site during therapy and automatically shut off the energy delivery when the temperature reaches a predetermined maximum (e.g., when applying RF energy) or predetermined minimum (e.g., when applying cryotherapy). In other embodiments, the evaluation/feedback algorithm 110, in conjunction with the controller 107, can be configured to automatically terminate treatment after a predetermined maximum time, a predetermined maximum impedance rise of the targeted tissue (i.e., in comparison to a baseline impedance measurement), a predetermined maximum impedance of the targeted tissue, and/or other threshold values for biomarkers associated with autonomic function. This and other information associated with the operation of the system 100 can be communicated to the operator via a graphical user interface (GUI) 112 provided via a display on the console 104 and/or a separate display (not shown) communicatively coupled to the console 104, such as a tablet or monitor, to thereby provide visual and/or audible alerts to the operator. The GUI 112 may generally provide operational instructions for the procedure, such as directing the operator to select which sino-nasal cavity to treat, indicating when the device 102 is primed and ready to perform treatment, and further providing status of therapy during the procedure, including indicating when the treatment is complete.

For example, in some embodiments, the end effector 114 and/or other portions of the system 100 can be configured to detect various parameters of the heterogeneous tissue at the target site to determine the anatomy at the target site (e.g., tissue types, tissue locations, vasculature, bone structures, foramen, sinuses, etc.), locate nerves and/or other structures, and allow for neural mapping. For example, the end effector 114 may be configured to detect impedance, dielectric properties, temperature, and/or other properties that indicate the presence of neural fibers in the target region.

As shown in FIG. 1A, the console 104 further includes a monitoring system 108 configured to receive data from the end effector 114 (i.e., detected electrical and/or thermal measurements of tissue at the target site), specifically sensed by appropriate sensors (e.g., temperature sensors and/or impedance sensors, or the like), and process this information to identify the presence of nerves, the location of nerves, neural activity at the target site, and/or other properties of the neural tissue, such a physiological properties (e.g., depth), bioelectric properties, and thermal properties. The nerve monitoring system 108 can be operably coupled to the electrodes and/or other features of the end effector 114 via signal wires (e.g., copper wires) that extend through the cable 120 and through the length of the shaft 116. In other embodiments, the end effector 114 can be communicatively coupled to the nerve monitoring system 108 using other suitable communication means.

The nerve monitoring system 108 can determine neural locations and activity before therapeutic neuromodulation to determine precise treatment regions corresponding to the positions of the desired nerves. The nerve monitoring system 108 can further be used during treatment to determine the effect of the therapeutic neuromodulation, and/or after treatment to evaluate whether the therapeutic neuromodulation treated the target nerves to a desired degree. This information can be used to make various determinations related to the nerves proximate to the target site, such as whether the target site is suitable for neuromodulation. In addition, the nerve monitoring system 108 can also compare the detected neural locations and/or activity before and after therapeutic neuromodulation, and compare the change in neural activity to a predetermined threshold to assess whether the application of therapeutic neuromodulation was effective across the treatment site. For example, the nerve monitoring system 108 can further determine electroneurogram (ENG) signals based on recordings of electrical activity of neurons taken by the end effector 114 before and after therapeutic neuromodulation. Statistically meaningful (e.g., measurable or noticeable) decreases in the ENG signal(s) taken after neuromodulation can serve as an indicator that the nerves were sufficiently ablated. Additional features and functions of the nerve monitoring system 108, as well as other functions of the various components of the console 104, including the evaluation/feedback algorithms 110 for providing real-time feedback capabilities for ensuring optimal therapy for a given treatment is administered, are described in at least U.S. Publication No. 2016/0331459 and U.S. Publication No. 2018/0133460, the contents of each of which are incorporated by reference herein in their entireties.

The device 102 provides access to target sites associated with peripheral nerves for the subsequent neuromodulation of such nerves and treatment of a corresponding peripheral neurological condition or disorder. The peripheral nervous system is one of two components that make up the nervous system of bilateral animals, with the other part being the central nervous system (CNS). The PNS consists of the nerves and ganglia outside the brain and spinal cord. The main function of the PNS is to connect the CNS to the limbs and organs, essentially serving as a relay between the brain and spinal cord and the rest of the body. The peripheral nervous system is divided into the somatic nervous system and the autonomic nervous system. In the somatic nervous system, the cranial nerves are part of the PNS with the exception of the optic nerve (cranial nerve II), along with the retina. The second cranial nerve is not a true peripheral nerve but a tract of the diencephalon. Cranial nerve ganglia originated in the CNS. However, the remaining ten cranial nerve axons extend beyond the brain and are therefore considered part of the PNS. The autonomic nervous system exerts involuntary control over smooth muscle and glands. The connection between CNS and organs allows the system to be in two different functional states: sympathetic and parasympathetic. Accordingly, the devices, systems, and methods of the present invention are useful in detecting, identifying, and precision targeting nerves associated with the peripheral nervous system for treatment of corresponding peripheral neurological conditions or disorders.

The peripheral neurological conditions or disorders may include, but are not limited to, chronic pain, movement disorders, epilepsy, psychiatric disorders, cardiovascular disorders, gastrointestinal disorders, genitourinary disorders, to name a few. For example, chronic pain may include headaches, complex regional pain syndrome, neuropathy, peripheral neuralgia, ischemic pain, failed back surgery syndrome, and trigeminal neuralgia. The movement disorders may include spasticity, Parkinson's disease, tremor, dystonia, Tourette syndrome, camptocormia, hemifacial spasm, and Meige syndrome. The psychiatric disorders may include depression, obsessive compulsive disorder, drug addiction, and anorexia/eating disorders. The functional restoration may include restoration of certain functions post traumatic brain injury, hearing impairment, and blindness. The cardiovascular disorders may include angina, heart failure, hypertension, peripheral vascular disorders, and stroke. The gastrointestinal disorders may include dysmotility and obesity. The genitourinary disorders may include painful bladder syndrome, interstitial cystitis, and voiding dysfunction.

For example, the system 100 may be used for the treatment of a cardiovascular disorder, such as arrhythmias or heart rhythm disorders, including, but not limited to, atrial fibrillation (AF or A-fib). Atrial fibrillation is an irregular and often rapid heart rate that can increase one's risk of stroke, heart failure, and other heart-related complications. Atrial fibrillation occurs when regions of cardiac tissue abnormally conduct electric signals to adjacent tissue, thereby disrupting the normal cardiac cycle and causing asynchronous rhythm. Atrial fibrillation symptoms often include heart palpitations, shortness of breath, and weakness. While episodes of atrial fibrillation can come and go, a person may develop atrial fibrillation that doesn't go away and thus will require treatment. Although atrial fibrillation itself usually isn't life-threatening, it is a serious medical condition that sometimes requires emergency treatment, as it may lead to complications. For example, atrial fibrillation is associated with an increased risk of heart failure, dementia, and stroke.

The normal electrical conduction system of the heart allows the impulse that is generated by the sinoatrial node (SA node) of the heart to be propagated to and stimulate the myocardium (muscular layer of the heart). When the myocardium is stimulated, it contracts. It is the ordered stimulation of the myocardium that allows efficient contraction of the heart, thereby allowing blood to be pumped to the body. In AF, the normal regular electrical impulses generated by the sinoatrial node in the right atrium of the heart are overwhelmed by disorganized electrical impulses usually originating in the roots of the pulmonary veins. This leads to irregular conduction of ventricular impulses that generate the heartbeat. In particular, during AF, the heart's two upper chambers (the atria) beat chaotically and irregularly, out of coordination with the two lower chambers (the ventricles) of the heart.

During atrial fibrillation, the regular impulses produced by the sinus node for a normal heartbeat are overwhelmed by rapid electrical discharges produced in the atria and adjacent parts of the pulmonary veins. Sources of these disturbances are either automatic foci, often localized at one of the pulmonary veins, or a small number of localized sources in the form of either a re-entrant leading circle, or electrical spiral waves (rotors). These localized sources may be found in the left atrium near the pulmonary veins or in a variety of other locations through both the left or right atrium. There are three fundamental components that favor the establishment of a leading circle or a rotor: 1) slow conduction velocity of cardiac action potential; 2) short refractory period; and 3) small wavelength. Wavelength is the product of velocity and refractory period. If the action potential has fast conduction, with a long refractory period and/or conduction pathway shorter than the wavelength, an AF focus would not be established. In multiple wavelet theory, a wavefront will break into smaller daughter wavelets when encountering an obstacle, through a process called vortex shedding; but under proper conditions, such wavelets can reform and spin around a center, forming an AF focus.

The system 100 provides for the treatment of AF, in which the device 102 may provide access to and provide treatment of one or more target sites associated with nerves that correspond to, or are otherwise associated with, treating AF. For example, the device 102, in conjunction with the console 104, may detect, identify, and precision target cardiac tissue and subsequently deliver energy at a level or frequency sufficient to therapeutically modulate nerves associated with such cardiac tissue. The therapeutic modulation of such nerves is sufficient to disrupt the origin of the signals causing the AF and/or disrupt the conducting pathway for such signals.

Similar to the conduction system of the heart, a neural network exists which surrounds the heart and plays an important role in formation of the substrate of AF and when a trigger is originated, usually from pulmonary vein sleeves, AF occurs. This neural network includes ganglionated plexi (GP) located adjacent to pulmonary vein ostia which are under control of higher centers in normal people. For example, the heart is richly innervated by the autonomic nerves. The ganglion cells of the autonomic nerves are located either outside the heart (extrinsic) or inside the heart (intrinsic). Both extrinsic and intrinsic nervous systems are important for cardiac function and arrhythmogenesis. The vagal nerves include axons that come from various nuclei in the medulla. The extrinsic sympathetic nerves come from the paravertebral ganglia, including the superior cervical ganglion, middle cervical ganglion, the cervicothoracic (stellate) ganglion and the thoracic ganglia. The intrinsic cardiac nerves are found mostly in the atria, and are intimately involved in atrial arrhythmogenesis cardiovascular disorder, such as arrhythmias or heart rhythm disorders, including, but not limited to, atrial fibrillation. When GP become hyperactive owing to loss of inhibition from higher centers (e.g., in elderly), AF can occur.

The system 100 can be used to control hyperactive GP either by stimulating higher centers and their connections, such as vagus nerve stimulation, or simply by ablating GP. Accordingly, the device 102, in conjunction with the console 104, may detect and identify ganglionated plexus (GP) and further determine an energy level sufficient to therapeutically modulate or treat (i.e., ablate) the GP for the treatment of AF (i.e., surgically disrupting the origin of the signals causing the AF and disrupting the conducting pathway for such signals) while minimizing and/or preventing collateral damage to surrounding or adjacent non-neural tissue including bloods vessels and bone and non-targeted neural tissue. It should be noted that other nerves and/or cardiac tissue, or other structures, known to have an impact on or cause AF, may be targeted by the system 100, including, but not limited to, pulmonary veins (e.g., pulmonary vein isolation upon creation of lesions around PV ostia to prevent triggers from reaching atrial substrate).

In addition to treating arrhythmias, the system 100 may also be used for the treatment of other cardiovascular-related conditions, particularly those involving the kidney. The kidneys play a significant role in the progression of CHF, as well as in Chronic Renal Failure (CRF), End-Stage Renal Disease (ESRD), hypertension (pathologically high blood pressure), and other cardio-renal diseases.

The functions of the kidney can be summarized under three broad categories: filtering blood and excreting waste products generated by the body's metabolism; regulating salt, water, electrolyte and acid-base balance; and secreting hormones to maintain vital organ blood flow. Without properly functioning kidneys, a patient will suffer water retention, reduced urine flow and an accumulation of waste toxins in the blood and body. These conditions resulting from reduced renal function or renal failure (kidney failure) are believed to increase the workload of the heart.

For example, in a CHF patient, renal failure will cause the heart to further deteriorate as the water build-up and blood toxins accumulate due to the poorly functioning kidneys and, in turn, cause the heart further harm. CHF is a condition that occurs when the heart becomes damaged and reduces blood flow to the organs of the body. If blood flow decreases sufficiently, kidney function becomes impaired and results in fluid retention, abnormal hormone secretions and increased constriction of blood vessels. These results increase the workload of the heart and further decrease the capacity of the heart to pump blood through the kidney and circulatory system. This reduced capacity further reduces blood flow to the kidney. It is believed that progressively decreasing perfusion of the kidney is a principal non-cardiac cause perpetuating the downward spiral of CHF. Moreover, the fluid overload and associated clinical symptoms resulting from these physiologic changes are predominant causes for excessive hospital admissions, reduced quality of life, and overwhelming costs to the health care system due to CHF.

End-stage renal disease is another condition at least partially controlled by renal neural activity. There has been a dramatic increase in patients with ESRD due to diabetic nephropathy, chronic glomerulonephritis and uncontrolled hypertension. Chronic renal failure (CRF) slowly progresses to ESRD. CRF represents a critical period in the evolution of ESRD. The signs and symptoms of CRF are initially minor, but over the course of 2-5 years, become progressive and irreversible. While some progress has been made in combating the progression to, and complications of, ESRD, the clinical benefits of existing interventions remain limited.

Arterial hypertension is a major health problem worldwide. Treatment-resistant hypertension is defined as the failure to achieve target blood pressure despite the concomitant use of maximally tolerated doses of three different antihypertensive medications, including a diuretic. Treatment-resistant hypertension is associated with considerable morbidity and mortality. Patients with treatment-resistant hypertension have markedly increased cardiovascular morbidity and mortality, facing an increase in the risk of myocardial infarction (MI), stroke, and death compared to patients whose hypertension is adequately controlled.

The autonomic nervous system is recognized as an important pathway for control signals that are responsible for the regulation of body functions critical for maintaining vascular fluid balance and blood pressure. The autonomic nervous system conducts information in the form of signals from the body's biologic sensors such as baroreceptors (responding to pressure and volume of blood) and chemoreceptors (responding to chemical composition of blood) to the central nervous system via its sensory fibers. It also conducts command signals from the central nervous system that control the various innervated components of the vascular system via its motor fibers.

It is known from clinical experience and research that an increase in renal sympathetic nerve activity leads to vasoconstriction of blood vessels supplying the kidney, decreased renal blood flow, decreased removal of water and sodium from the body, and increased renin secretion. It is also known that reduction of sympathetic renal nerve activity, e.g., via denervation, may reverse these processes.

The renal sympathetic nervous system plays a critical influence in the pathophysiology of hypertension. The adventitia of the renal arteries has efferent and afferent sympathetic nerves. Renal sympathetic activation via the efferent nerves initiates a cascade resulting in elevated blood pressure. Efferent sympathetic outflow leads to vasoconstriction with a subsequent reduction in glomerular blood flow, a lowering of the glomerular filtration rate, release of renin by the juxtaglomerular cells, and the subsequent activation of the renin-angiotensin-aldosterone axis leading to increased tubular reabsorption of sodium and water. Decreased glomerular filtration rate also prompts additional systemic sympathetic release of catecholamines. As a consequence, blood pressure increases by a rise in total blood volume and increased peripheral vascular resistance.

The system 100 can be used for the treatment of cardio-renal diseases, including hypertension, by providing renal neuromodulation and/or denervation. For example, the device 102 may be placed at one or more target sites associated with renal nerves other neural fibers that contribute to renal neural function, or other neural features. For example, the device 102, in conjunction with the console 104, may detect, identify, and precision target renal nerve tissue and subsequently deliver energy at a level or frequency sufficient to therapeutically modulate nerves associated with such renal tissue. The therapeutic modulation of such renal nerves and/or renal tissue is sufficient to completely block or denervate the target neural structures and/or disrupt renal nervous activity, while minimizing and/or preventing collateral damage to surrounding or adjacent non-neural tissue including bloods vessels and bone and non-targeted neural tissue.

It should further be noted that the system 100 can be used to determine disease progression. In particular, the present system 100 can obtain measurements at one or more target sites associated with a given disease, disorder, or the like. Such measurements may be based on the active neural parameters (i.e., neuronal firing and active voltage monitoring) and may be used to identify neurons. The active neural parameters (and thus behavior) change with disease progression, thereby allowing the present system to identify such changes and determine a progression of the underlying disease or disorder. Such capabilities are possible based, at least in part, on the fact that the present system 100 is configured to monitor passive electric phenomena (i.e., the present system 100 determines the ohmic conductivity frequency, which remains consistent, while conductivity will be different based on disease or disorder progression).

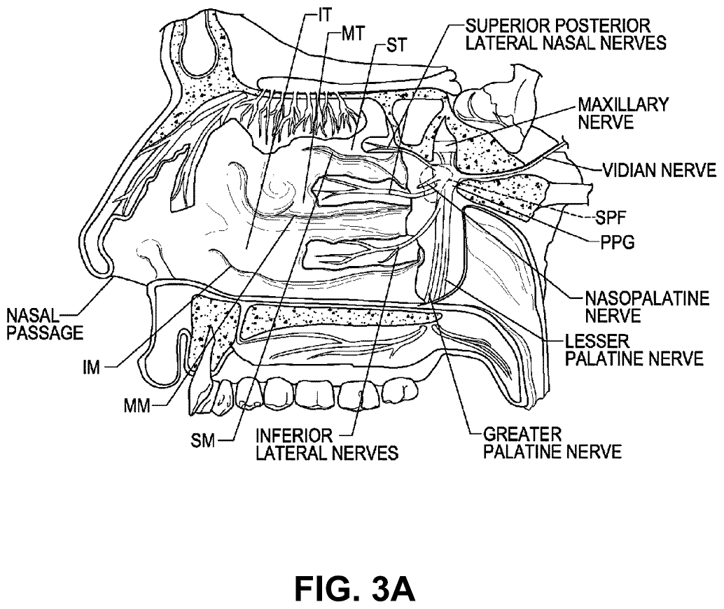

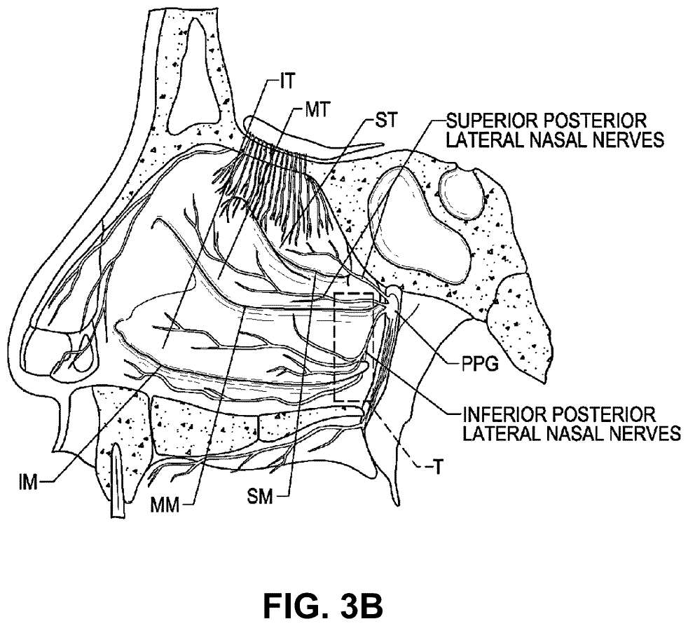

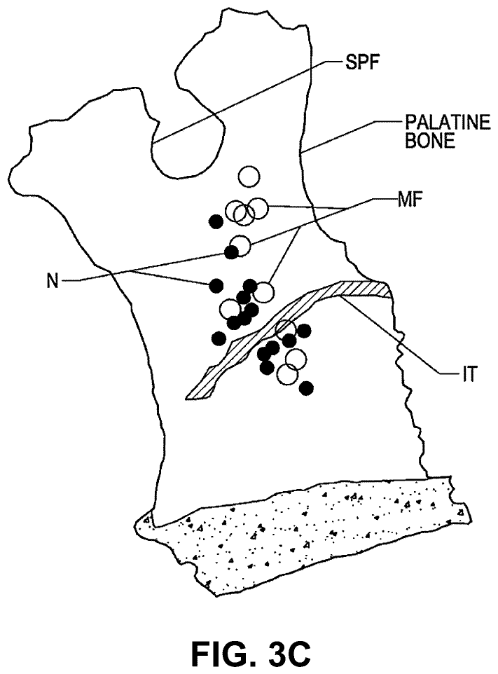

FIG. 3A is a cut-away side view illustrating the anatomy of a lateral sino-nasal wall and FIG. 3B is an enlarged side view of the nerves of the lateral sino-nasal wall of FIG. 1A. The sphenopalatine foramen (SPF) is an opening or conduit defined by the palatine bone and the sphenoid bone through which the sphenopalatine vessels and the posterior superior sino-nasal nerves travel into the sino-nasal cavity. More specifically, the orbital and sphenoidal processes of the perpendicular plate of the palatine bone define the sphenopalatine notch, which is converted into the SPF by the articulation with the surface of the body of the sphenoid bone.

The location of the SPF is highly variable within the posterior region of the lateral sino-nasal cavity, which makes it difficult to visually locate the SPF. Typically, the SPF is located in the middle meatus (MM). However, anatomical variations also result in the SPF being located in the superior meatus (SM) or at the transition of the superior and middle meatuses. In certain individuals, for example, the inferior border of the SPF has been measured at about 19 mm above the horizontal plate of the palatine bone (i.e., the sino-nasal sill), which is about 13 mm above the horizontal lamina of the inferior turbinate (IT) and the average distance from the sino-nasal sill to the SPF is about 64.4 mm, resulting in an angle of approach from the sino-nasal sill to the SPA of about 11.4°. However, studies to measure the precise location of the SPF are of limited practical application due to the wide variation of its location.

The anatomical variations of the SPF are expected to correspond to alterations of the autonomic and vascular pathways traversing into the sino-nasal cavity. In general, it is thought that the posterior sino-nasal nerves (also referred to as lateral posterior superior sino-nasal nerves) branch from the pterygopalatine ganglion (PPG), which is also referred to as the sphenopalatine ganglion (SPG), through the SPF to enter the lateral sino-nasal wall of the sino-nasal cavity, and the sphenopalatine artery passes from the pterygopalatine fossa through the SPF on the lateral sino-nasal wall. The sphenopalatine artery branches into two main portions: the posterior lateral sino-nasal branch and the posterior septal branch. The main branch of the posterior lateral sino-nasal artery travels inferiorly into the inferior turbinate IT (e.g., between about 1.0 mm and 1.5 mm from the posterior tip of the inferior turbinate IT), while another branch enters the middle turbinate MT and branches anteriorly and posteriorly.

Beyond the SPF, studies have shown that over 30% of human patients have one or more accessory foramen that also carries arteries and nerves into the sino-nasal cavity. The accessory foramen are typically smaller than the SPF and positioned inferior to the SPF. For example, there can be one, two, three or more branches of the posterior sino-nasal artery and posterior sino-nasal nerves that extend through corresponding accessory foramen. The variability in location, size, and quantity associated with the accessory foramen and the associated branching arteries and nerves that travel through the accessory foramen gives rise to a great deal of uncertainty regarding the positions of the vasculature and nerves of the sphenopalatine region. Furthermore, the natural anatomy extending from the SPF often includes deep inferior and/or superior grooves that carry neural and arterial pathways, which make it difficult to locate arterial and neural branches. For example the grooves can extend more than 5 mm long, more than 2 mm wide, and more than 1 mm deep, thereby creating a path significant enough to carry both arteries and nerves. The variations caused by the grooves and the accessory foramen in the sphenopalatine region make locating and accessing the arteries and nerves (positioned posterior to the arteries) extremely difficult for surgeons.

Recent microanatomic dissection of the pterygopalatine fossa (PPF) have further evidenced the highly variable anatomy of the region surrounding the SPF, showing that a multiplicity of efferent rami that project from the pterygopalatine ganglion (PPG) to innervate the orbit and sino-nasal mucosa via numerous groups of small nerve fascicles, rather than an individual postganglionic autonomic nerves (e.g., the posterior sino-nasal nerve). Studies have shown that at least 87% of humans have microforamina and micro rami in the palatine bone.

FIG. 3C, for example, is a front view of a left palatine bone illustrating geometry of microforamina and micro rami in a left palatine bone. In FIG. 3C, the solid regions represent nerves traversing directly through the palatine bone, and the open circles represent nerves that were associated with distinct microforamina. As such, FIG. 3C illustrates that a medial portion of the palatine bone can include at least 25 accessory posterolateral nerves.

The respiratory portion of the sino-nasal cavity mucosa is composed of a type of ciliated pseudostratified columnar epithelium with a basement membrane. sino-nasal secretions (e.g., mucus) are secreted by goblet cells, submucosal glands, and transudate from plasma. sino-nasal seromucous glands and blood vessels are highly regulated by parasympathetic innervation deriving from the vidian and other nerves. Parasympathetic (cholinergic) stimulation through acetylcholine and vasoactive intestinal peptide generally results in mucus production. Accordingly, the parasympathetic innervation of the mucosa is primarily responsible submucosal gland activation/hyper activation, venous engorgement (e.g., congestion), and increased blood flow to the blood vessels lining the nose. Accordingly, severing or modulating the parasympathetic pathways that innervate the mucosa are expected to reduce or eliminate the hyper activation of the submucosal glands and engorgement of vessels that cause symptoms associated with rhinosinusitis and other indications.

As previously described herein, postganglionic parasympathetic fibers that innervate the sino-nasal mucosa (i.e., posterior superior sino-nasal nerves) were thought to travel exclusively through the SPF as a sphenopalatine neurovascular bundle. The posterior sino-nasal nerves are branches of the maxillary nerve that innervate the sino-nasal cavity via a number of smaller medial and lateral branches extending through the mucosa of the superior and middle turbinates ST, MT (i.e., sino-nasal conchae) and to the sino-nasal septum. The nasopalatine nerve is generally the largest of the medial posterior superior sino-nasal nerves, and it passes anteroinferiorly in a groove on the vomer to the floor of the sino-nasal cavity. From here, the nasopalatine nerve passes through the incisive fossa of the hard palate and communicates with the greater palatine nerve to supply the mucosa of the hard palate. The posterior superior sino-nasal nerves pass through the pterygopalatine ganglion PPG without synapsing and onto the maxillary nerve via its ganglionic branches.

Based on the understanding that the posterior sino-nasal nerves exclusively traverse the SPF to innervate the sino-nasal mucosa, surgeries have been performed to selectively sever the posterior sino-nasal nerve as it exits the SPF. However, as discussed above, the sinonasal parasympathetic pathway actually comprises individual rami project from the pterygopalatine ganglion (PPG) to innervate the sino-nasal mucosa via multiple small nerve fascicles (i.e., accessory posterolateral nerves), not a single branch extending through the SPF. These rami are transmitted through multiple fissures, accessory foramina, and microforamina throughout the palatine bone and may demonstrate anastomotic loops with both the SPF and other accessory nerves. Thus, if only the parasympathetic nerves traversing the SPF were severed, almost all patients (e.g., 90% of patients or more) would retain intact accessory secretomotor fibers to the posterolateral mucosa, which would result in the persistence of symptoms the neurectomy was meant to relieve.

Accordingly, embodiments of the present disclosure are configured to therapeutically modulate nerves at precise and focused treatment sites corresponding to the sites of rami extending through fissures, accessory foramina, and microforamina throughout the palatine bone (e.g., target region T shown in FIG. 3B). In certain embodiments, the targeted nerves are postganglionic parasympathetic nerves that go on to innervate the sino-nasal mucosa. This selective neural treatment is also expected to decrease the rate of postoperative sino-nasal crusting and dryness because it allows a clinician to titrate the degree of anterior denervation through judicious sparing of the rami orbitonasal. Furthermore, embodiments of the present disclosure are also expected to maintain at least some sympathetic tone by preserving a portion of the sympathetic contributions from the deep petrosal nerve and internal maxillary periarterial plexus, leading to improved outcomes with respect to sino-nasal obstruction. In addition, embodiments of the present disclosure are configured to target a multitude of parasympathetic neural entry locations (e.g., accessory foramen, fissures, and microforamina) to the sino-nasal region to provide for a complete resection of all anastomotic loops, thereby reducing the rate of long-term re-innervation.

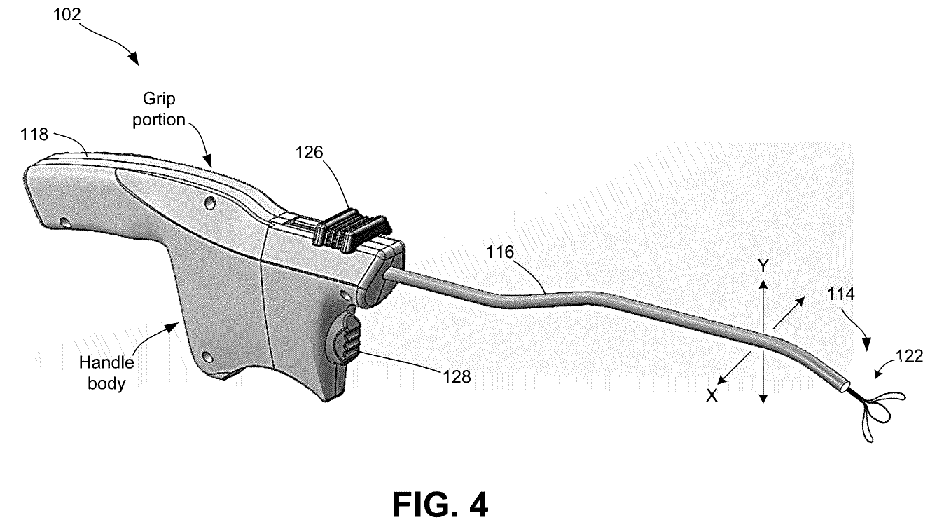

FIG. 4 is a side view of one embodiment of a handheld device for providing therapeutic neuromodulation consistent with the present disclosure. As illustrated, the device 102 includes an end effector 114 transformable between a retracted configuration and an expanded deployed configuration, a shaft 116 operably associated with the end effector 114, and a handle 118 operably associated with the shaft 116. As will be described in greater detail herein, the end effector 114 may include a single segment or may be multi-segmented, in which two or more segments may be provided. For example, in some embodiments, the end effector is comprised of a single segment, while, in other embodiments, the end effector is comprised of two segments (including at least a first segment and a second segment spaced apart from one another).

In any given embodiment, each segment of the end effector is transformable between a retracted configuration, which includes a low-profile delivery state to facilitate intraluminal delivery of the end effector 114 to a treatment site within the sino-nasal region, and a deployed configuration, which includes an expanded state (as shown in FIG. 4). The handle 118 may include at least a first mechanism 126 for deployment of the end effector, notably a single segment or the multiple segments, from the retracted configuration to the deployed configuration and a second mechanism 128, separate from the first mechanism 126, for control of energy output from energy delivering elements of a given segment, specifically electrodes or other energy elements provided by a given segment.

For example, the first mechanism 126 may include a slider mechanism with overhang to allow easier interaction with index/middle finger so that user can move the outer sheath to change the end effector from a retracted to expanded configuration. The second mechanism 128 may include button or trigger mechanism on the front end of the handle body, which facilitates user input so that selections on the console interface can be made and energy delivery can be initiated from the RF generator to the end effector. It should further be noted that the device may include a larger stock on handle body, which optimizes user interaction with the device and facilitates a more intuitive underhand grip. Furthermore, the shaft 116 may be malleable and can be manipulated by user so as to deform it in multiple directions (i.e., at least X- and Y-axes, as well as a Z-axis) to thereby facilitate improved contact between the end effector and tissue sites in a given nasal cavity.

As previously described herein, the underlying design of the end effector 114 is unique. For example, in one embodiment, the end effector 114 includes a single segment having a bilateral geometry. In particular, the single segment includes two identical sides, including a first side formed of struts and a second side formed of struts. This bilateral geometry allows at least one of the two sides to conform to and accommodate an anatomical structure within the sino-nasal cavity when the segment in an expanded state. For example, when in the expanded state, the plurality of struts contact multiple locations along multiple portions of the anatomical structure and electrodes provided by the struts are configured to emit energy at a level sufficient to create multiple micro-lesions in tissue of the anatomical structure that interrupt neural signals to mucus producing and/or mucosal engorgement elements.

For example, the bilateral geometry refers to the fact that segments of one side of the end effector accommodate various anatomical locations along the lateral wall of one nasal cavity (i.e., left nasal cavity), while segments on the opposing side of the end effector accommodate locations on the lateral wall within the other nasal cavity (i.e., right nasal cavity). It should be noted that, in other embodiments, the end effector is multi-segmented in that it is composed of two or more segments, each having a bilateral geometry as previously described. Furthermore, in other embodiments, the end effector may include a single segment or multiple segments, each having a unilateral geometry.

The advantage of the single stage bilateral geometry is that it is simpler from a design and cost perspective and can be used to treat multiple locations in the middle meatus and posterior to the middle turbinate along the lateral wall of each nasal cavity.

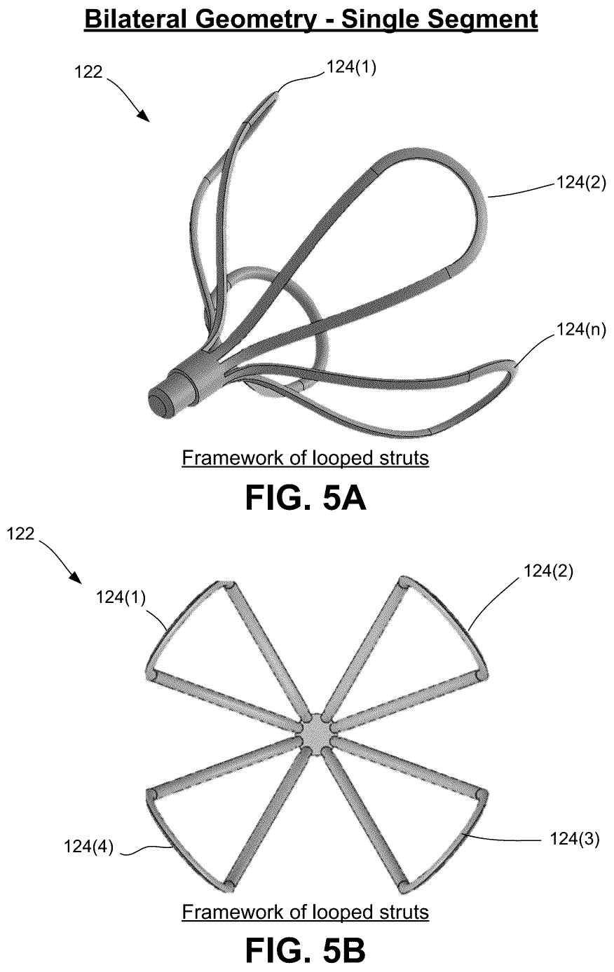

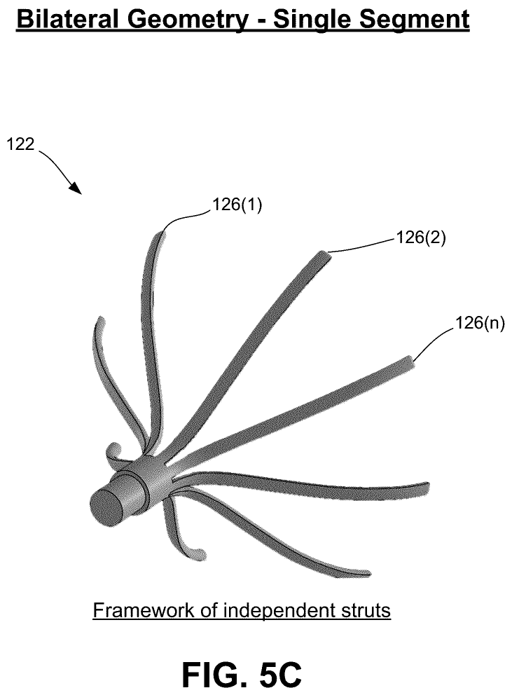

FIGS. 5A and 5B are perspective and front (proximal facing) views, respectively, of one embodiment of a single segment 122 having a bilateral geometry, illustrating a framework of loop-shaped struts 124(1)-124(n) of the single segment 122. FIG. 5C is a perspective view of another embodiment of a single segment 122 having a bilateral geometry, illustrating a framework of independent struts 126(1)-126(n) of the single segment 122.

The support elements or struts are generally in the form of wires that have elastic properties. For example, in some embodiments, the struts may include a shape memory material, such as nitinol. The flexible support elements may further include a highly lubricious coating, which may allow for desirable electrical insulation properties as well as desirable low friction surface finish. Each of the struts is transformable between a retracted configuration and an expanded deployed configuration such that the struts are configured to position one or more electrodes provided thereon into contact with one or more target sites when in the deployed configuration.

It should be noted that the struts generally serve as an underlying framework upon which energy delivering elements (i.e., electrodes or the like) can be positioned. For example, as previously noted, the end effector of the present invention may further include one or more flexible printed circuit board (PCB) members operably associated therewith. Accordingly, as will be described in greater detail herein, any given segment of the end effector essentially serves as a framework upon which separate respective flexible PCB assemblies are attached, wherein energy delivering elements, such as electrodes, are provided via flexible PCB members.

In this manner, the present invention provides an end effector that is capable of highly conforming to anatomical variations within a sino-nasal cavity so that an operator can perform an accurate, minimally invasive, and localized application of energy to one or more target sites within the sino-nasal cavity of the patient to thereby treat a sino-nasal condition. In particular, unlike other surgical treatments for rhinitis, the devices of the invention are minimally invasive. Once delivered within the sino-nasal cavity, each segment of the end effector can expand to a specific shape and/or size corresponding to anatomical structures within the sino-nasal cavity and associated with the target sites. A plurality of flexible PCB members attached to a given segment of the end effector are able to correspondingly move and transition into the specific geometry of the given segment, such that, once deployed, the flexible PCBs contact and conform to a shape of the respective locations, including conforming to and complementing shapes of one or more anatomical structures at the respective locations.

In turn, the plurality of flexible PCB members become accurately positioned within the sino-nasal cavity to subsequently deliver, via one or more electrodes, precise and focused application of energy to targeted tissue at the one or more target sites, to disrupt multiple neural signals to, and/or result in local hypoxia of, mucus producing and/or mucosal engorgement elements, thereby reducing production of mucus and/or mucosal engorgement within a nose of the patient and reducing or eliminate one or more symptoms associated with at least one of rhinitis, congestion, and rhinorrhea.

The use of flexible PCBs members results in a greater amount of usable surface area than what is otherwise available with existing end effectors. In particular, the increase in surface area allows for a greater number of energy delivery elements to be introduced and utilized in a given procedure and further expands the possible number of patterns of such energy delivery elements. As a result, the contact surface increases substantially, thereby allowing for the end effector of the present invention to deliver treatment to certain areas within the sino-nasal cavity that may have been previously unreachable or untreatable with current treatment devices, or that previously required a surgeon to reposition a given device to reach such areas. Furthermore, the use of flexible PCB members reduces the overall complexity with regard to manufacturing the end effector of the present invention. In particular, any given flexible PCB member (including an overall PCB assembly, which includes multiple PCB members) is constructed separately from the end effector, which includes constructed the overall electrode design and placement on a given PCB member. Once a PCB assembly is complete, PCB members are then attached to respective portions of a given segment of the end effector as a separate manufacturing step, thereby reducing the complexity that is otherwise associated with placing electrodes directly on the end effector, which is a common practice. It should be noted, however, that, in some embodiments, a given flexible PCB member can be integrated into a molded segment during manufacture.

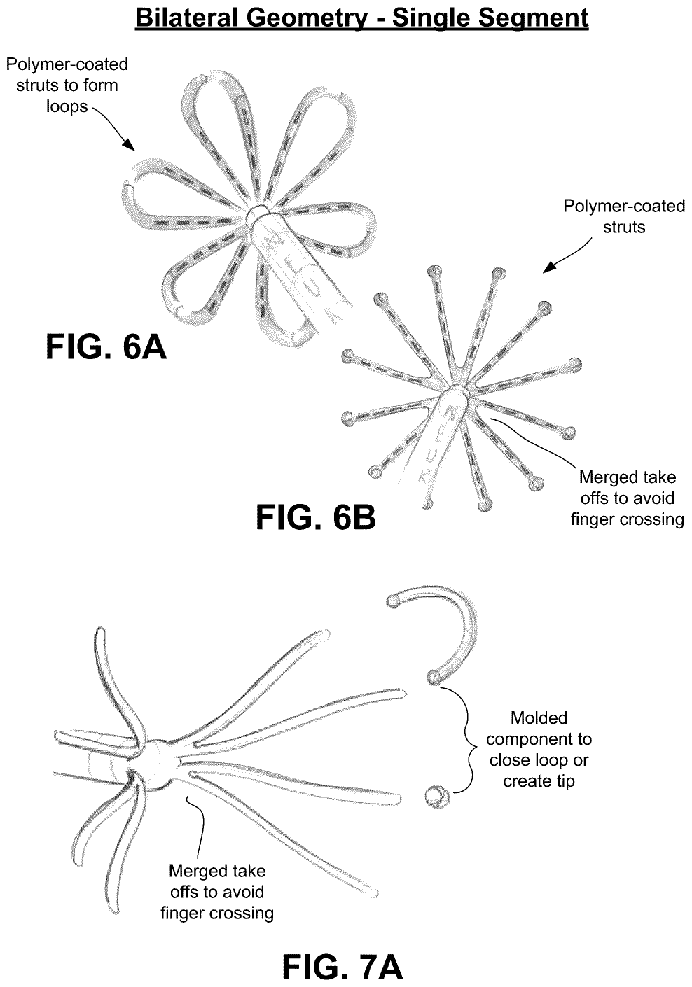

FIGS. 6A and 6B are perspective views of embodiments of a single segment that comprises of looped-shaped struts (FIG. 6A) and independent struts (FIG. 6B), respectively, in an expanded, deployed configuration and including a flexible printed circuit board (PCB) assembly thereon.

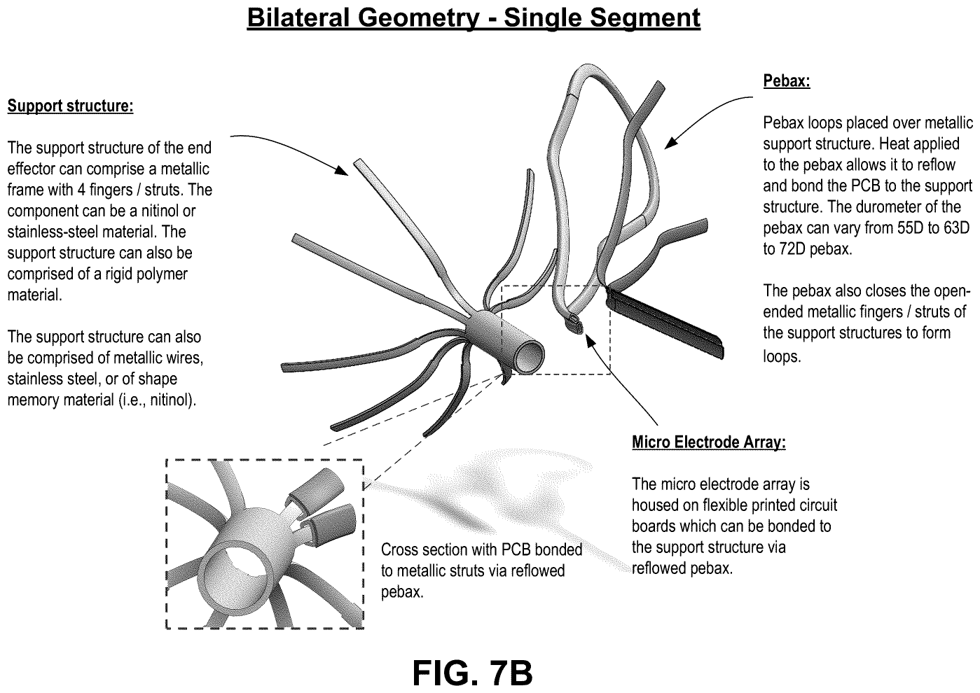

FIG. 7A is a perspective view illustrating placement of polymer caps or tubing to the free, distal ends of the struts to create atraumatic tips or close a loop and couple two struts to one another, respectively.

FIG. 7B is a perspective exploded view illustrating the interaction and assembly of components with one another in an exemplary embodiment of a single segment having a bilateral geometry.

As shown, when in the expanded deployed configuration, extend outward from a central axis to form an open-ended circumferential shape. In particular, the open-ended circumferential shape of the single segment shown in FIG. 6A generally resembles a blooming flower, wherein each looped strut may generally resemble a flower petal or leaflet. It should be noted that a given segment may include any number of individual struts and is not limited to the number shown. For example, in some embodiments, the segment may include approximately six looped struts (shown in FIG. 6A, in which pairs of immediately adjacent struts are joined via tubing or the like) or twelve individual, unconnected struts (shown in FIG. 6B). However, as shown in FIG. 9A, the segment may include approximately four looped struts, or, as shown in FIG. 9B, eight individual, unconnected struts.

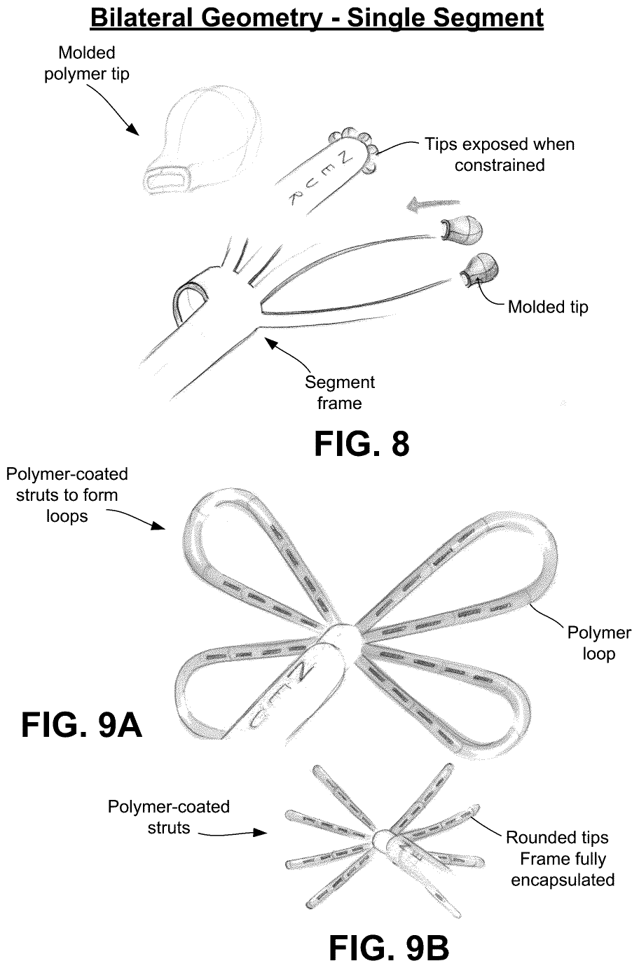

FIG. 8 is a perspective view illustrating loading of molded polymer tips to free distal ends of independent struts. The inclusion of such tips prevents potential trauma to the anatomy during use.

FIGS. 9A and 9B are perspective views of other embodiments of a single segment that comprises of looped-shaped struts (FIG. 9A) and independent struts (FIG. 9B), respectively, in an expanded, deployed configuration and including a flexible printed circuit board (PCB) assembly thereon.

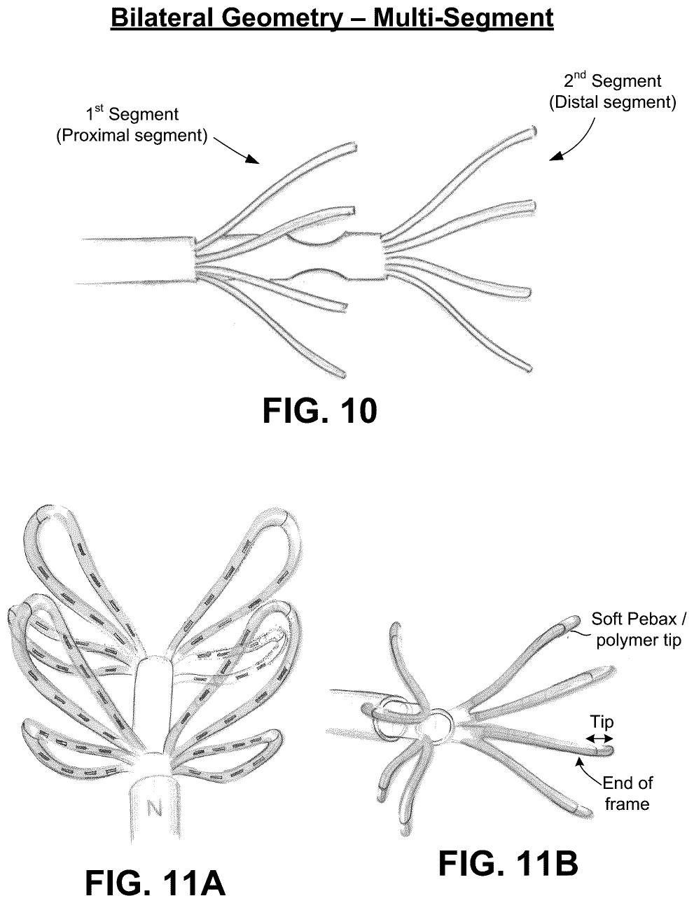

FIG. 10 is a side view of one embodiment of a multi-segment end effector composed of two separate segments (first and second segments) having a bilateral geometry, specifically illustrating the unitary construction of the framework of each.

FIGS. 11A and 11B are perspective views of embodiments of a multi-segment end effector in which each segment is comprised of looped-shaped struts (FIG. 11A) and independent struts (FIG. 11B), respectively, in an expanded, deployed configuration and including a flexible printed circuit board (PCB) assembly thereon.

It should be noted that, in some embodiments, the struts can be equidistantly spaced apart, while, in other embodiments, the struts can be arranged in a specific configuration about the central axis based on the specific location within the nasal cavity and the intended target site.

When in the expanded state, the struts (looped or independent) can position any number of electrodes against tissue at a target site within the sino-nasal region. The electrodes can apply bipolar or multi-polar radiofrequency (RF) energy to the target site to therapeutically modulate postganglionic parasympathetic nerves that innervate the sino-nasal mucosa proximate to the target site. In various embodiments, the electrodes can be configured to apply pulsed RF energy with a desired duty cycle (e.g., 1 second on/0.5 seconds off) to regulate the temperature increase in the target tissue.

The struts can have sufficient rigidity to support the electrodes and position or press the electrodes against tissue at the target site. In addition, when in the expanded deployed configuration, the struts can press against surrounding anatomical structures proximate to the target site (e.g., the turbinates, the palatine bone, etc.) and at least partially conform to the shape of the adjacent anatomical structures to effectively anchor the end effector. In addition, the expansion and conformability of the struts can facilitate placing the electrodes in contact with the surrounding tissue at the target site.

In certain embodiments, each electrode can be operated independently of the other electrodes. For example, each electrode can be individually activated and the polarity and amplitude of each electrode can be selected by an operator or a control algorithm (e.g., executed by the controller 107 previously described herein). The selective independent control of the electrodes allows the end effector to deliver RF energy to highly customized regions. For example, a select portion of the electrodes can be activated to target neural fibers in a specific region while the other electrodes remain inactive. In certain embodiments, for example, electrodes may be activated across specific struts in a given segment (i.e., struts that are adjacent to, or otherwise in contact with, tissue at the target site), and the electrodes that are not proximate to the target tissue can remain inactive to avoid applying energy to non-target tissue. Such configurations facilitate selective therapeutic modulation of nerves on the lateral sino-nasal wall within one nostril without applying energy to structures in other portions of the sino-nasal cavity.

Once deployed, a given segment can conform to a shape of the respective locations, including conforming to and complementing shapes of one or more anatomical structures at the respective locations. In turn, a given segment becomes accurately positioned within the sino-nasal cavity to subsequently deliver, via one or more electrodes, precise and focused application of RF thermal energy to the one or more target sites to thereby therapeutically modulate associated neural structures. More specifically, any given segment (i.e., the single segment design or the two or more segments of the multi-segment design) have shapes and sizes when in the expanded configuration that are specifically designed to place portions of a given segment, and thus one or more electrodes associated therewith, into contact with target sites within sino-nasal cavity associated with postganglionic parasympathetic fibers that innervate the sino-nasal mucosa.

For example, portions of each segment may specifically complement a shape of one or more anatomical structures when the given segment is in the deployed configuration. The anatomical structures may include, but are not limited to, inferior turbinate, middle turbinate, superior turbinate, inferior meatus, middle meatus, superior meatus, pterygopalatine region, pterygopalatine fossa, sphenopalatine foramen, accessory sphenopalatine foramen(ae), and sphenopalatine micro-foramen(ae).

As illustrated, the segments of the single and multi-segment configurations comprise a bilateral geometry. In particular, each segment generally includes two identical sides, including a first side formed of one or more struts and a second side formed of one or more struts of equal number. This bilateral geometry allows at least one of the two sides to conform to and accommodate an anatomical structure within the sino-nasal cavity when the given segment is in an expanded state. For example, when in the expanded state, two sets of struts contact multiple locations along multiple portions of the anatomical structure and electrodes provided by the struts are configured to emit energy at a level sufficient to create multiple micro-lesions in tissue of the anatomical structure that interrupt neural signals to mucus producing and/or mucosal engorgement elements. For example, the bilateral geometry refers to the fact that segments of one side of the end effector accommodate various anatomical locations along the lateral wall of one nasal cavity (i.e., left nasal cavity), while segments on the opposing side of the end effector accommodate locations on the lateral wall within the other nasal cavity (i.e., right nasal cavity). It should be noted that, in other embodiments, the end effector is multi-segmented in that it is composed of two or more segments, each having a bilateral geometry as previously described. Furthermore, in other embodiments, the end effector may include a single segment or multiple segments, each having a unilateral geometry.

The advantage of the single stage bilateral geometry is that it is simpler from a design and cost perspective and can be used to treat multiple locations in the middle meatus and posterior to the middle turbinate along the lateral wall of each nasal cavity.

By having this independence between first and second side (i.e., right and left side) configurations, the segment is a true bilateral device. By providing a bilateral geometry, the multi-segment end effector does not require a repeat use configuration to treat the other side of the anatomical structure, as both sides of the structure are accounted at the same time due to the bilateral geometry. The resultant micro-lesion pattern can be repeatable and is predictable in both macro element (depth, volume, shape parameter, surface area) and can be controlled to establish low to high effects of each, as well as micro elements (the thresholding of effects within the range of the macro envelope can be controlled), as well be described in greater detail herein.

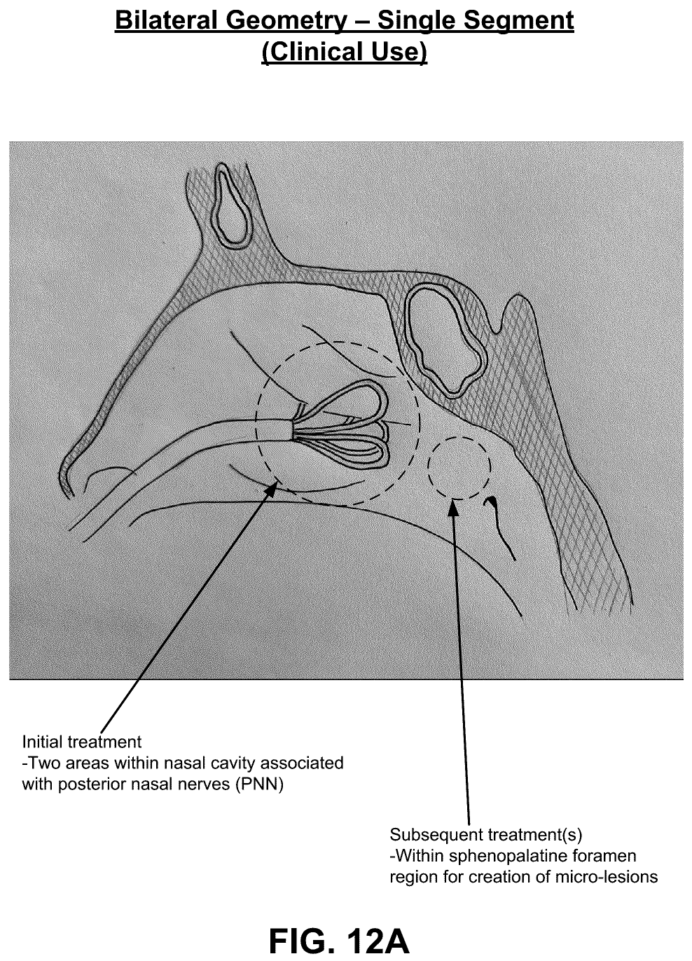

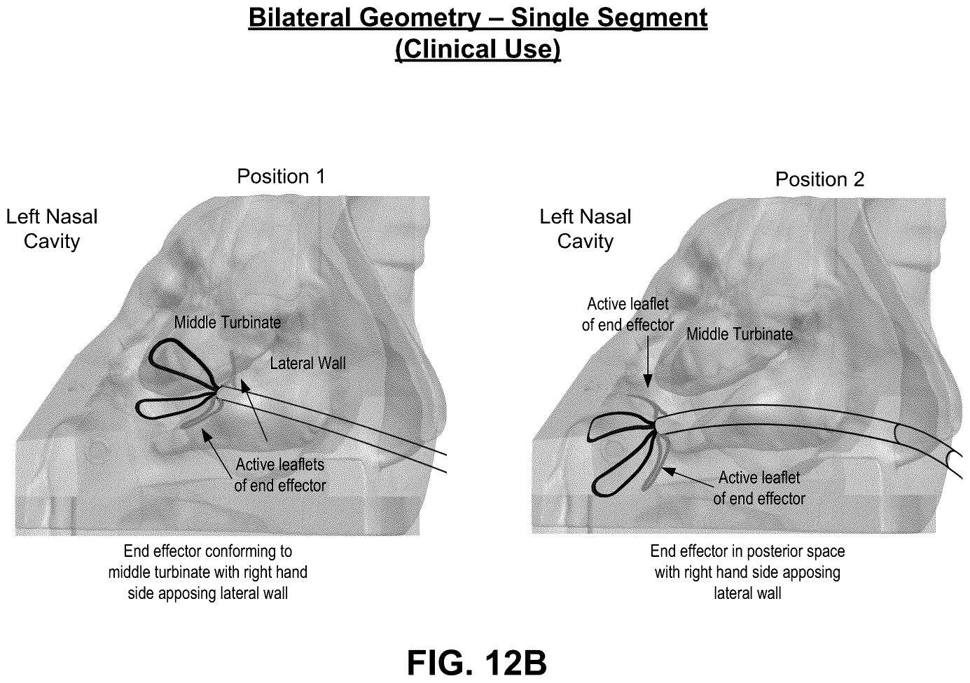

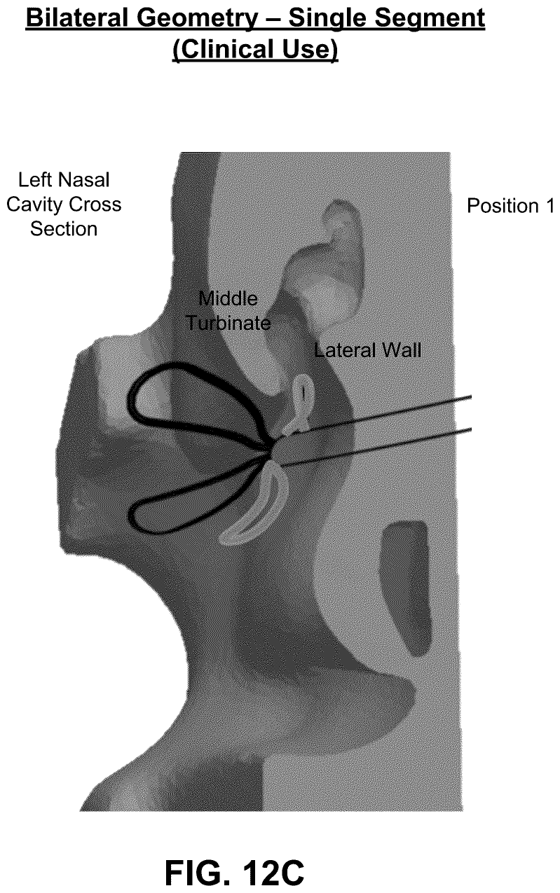

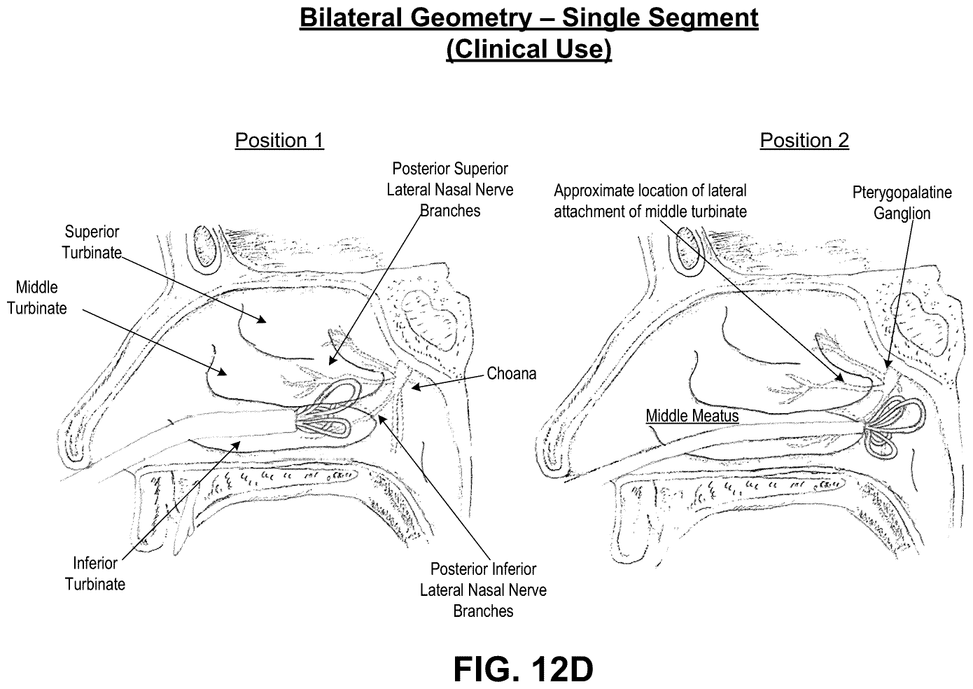

FIGS. 12A, 12B, and 12C are various views illustrating approaches for delivering a single segment end effector having a bilateral geometry to a target site within a nasal region in accordance with embodiments of the present disclosure.

As shown, the distal portion of the shaft extends into the nasal passage, where the end effector is deployed at a treatment site, specifically placed in two distinct areas of the nasal cavity associated with posterior nasal nerves (PNN). In particular, the end effector can be deployed and contact tissue sites at the lateral wall of the middle meatus. The single segment having a bilateral geometry allows for positioning of electrodes along the middle turbinate for multiple therapeutic energy delivery of the later wall. The end effector can then be used for subsequent treatments in the sphenopalatine foramen (SPF) region so that multiple micro lesions can be created along the lateral wall.

For example, once positioned at the target site, therapeutic modulation may be applied via the one or more electrodes and/or other features of the end effector to precise, localized regions of tissue to induce one or more desired therapeutic neuromodulating effects to disrupt parasympathetic motor sensory function. The end effector can selectively target postganglionic parasympathetic fibers that innervate the nasal mucosa at a target or treatment site proximate to or at their entrance into the nasal region.

For example, the end effector 114 can be positioned to apply therapeutic neuromodulation at least proximate to the SPF to therapeutically modulate nerves entering the nasal region via the SPF. The end effector 114 can also be positioned to inferior to the SPF to apply therapeutic neuromodulation energy across accessory foramen and microforamina (e.g., in the palatine bone) through which smaller medial and lateral branches of the posterior superior lateral nasal nerve enter the nasal region. The purposeful application of the energy at the target site may achieve therapeutic neuromodulation along all or at least a portion of posterior nasal neural fibers entering the nasal region. The therapeutic neuromodulating effects are generally a function of, at least in part, power, time, and contact between the energy delivery elements and the adjacent tissue. For example, in certain embodiments therapeutic neuromodulation of autonomic neural fibers are produced by applying RF energy at a power of about 2-20 W (e.g., 5 W, 7 W, 10 W, etc.) for a time period of about 1-20 sections (e.g., 5-10 seconds, 8-10 seconds, 10-12 seconds, etc.).

The therapeutic neuromodulating effects may include partial or complete denervation via thermal ablation and/or non-ablative thermal alteration or damage (e.g., via sustained heating and/or resistive heating). Desired thermal heating effects may include raising the temperature of target neural fibers above a desired threshold to achieve non-ablative thermal alteration, or above a higher temperature to achieve ablative thermal alteration. For example, the target temperature may be above body temperature (e.g., approximately 37° C.) but less than about 90° C. (e.g., 70-75° C.) for non-ablative thermal alteration, or the target temperature may be about 100° C. or higher (e.g., 110° C., 120° C., etc.) for the ablative thermal alteration. Desired non-thermal neuromodulation effects may include altering the electrical signals transmitted in a nerve.

Sufficiently modulating at least a portion of the parasympathetic nerves is expected to slow or potentially block conduction of autonomic neural signals to the nasal mucosa to produce a prolonged or permanent reduction in nasal parasympathetic activity. This is expected to reduce or eliminate activation or hyperactivation of the submucosal glands and venous engorgement and, thereby, reduce or eliminate the symptoms of rhinosinusitis. Further, because the device applies therapeutic neuromodulation to the multitude of branches of the posterior nasal nerves rather than a single large branch of the posterior nasal nerve branch entering the nasal cavity at the SPF, the device provides a more complete disruption of the parasympathetic neural pathway that affects the nasal mucosa and results in rhinosinusitis. Accordingly, the device is expected to have enhanced therapeutic effects for the treatment of rhinosinusitis and reduced re-innervation of the treated mucosa.

FIG. 12D provides additional views illustrating an approach for delivering a single segment end effector having a bilateral geometry to a target site within a nasal region in accordance with embodiments of the present disclosure, specifically illustrating the right nasal cavity. The lateral attachment of the middle turbinate varies in size and position. This results in difficult placement of electrodes in both the middle meatus and posterior position (choana) of the nasal cavity. It is therefore difficult to appose tissue sites simultaneously in both locations and deliver energy to primary parasympathetic nerve pathways.

The single stage design ensures full apposition of electrodes to tissue so as to target these nerve pathways in the middle meatus first (position 1), followed by full electrode apposition and targeting of nerve pathways in the region of the choana. This approach ensures effective de-enervation of the Posterior Superior Lateral Nasal Nerve Branches located in the lateral wall of the middle meatus, followed by de-enervation of inferior branches proximate to the pterygopalatine ganglion.

As previously noted herein, a given segment of the end effector may include a unilateral geometry (i.e., struts extending from one side of the end effector, as opposed to a bilateral geometry in which struts extend from opposing sides of the end effector). As will be described in greater detail herein, the unilateral design allows for targeting tissue in the middle meatus and posterior space.

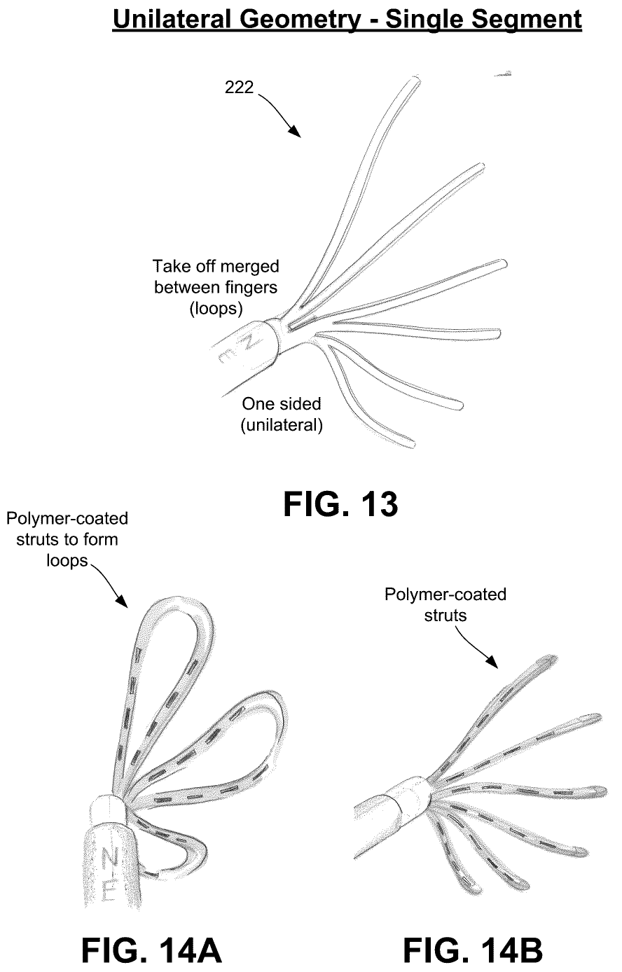

For example, FIG. 13 is a perspective view of one embodiment of a single segment having a unilateral geometry, illustrating a framework of independent struts of the single segment. FIGS. 14A and 14B are perspective views of embodiments of a single segment having a unilateral geometry that comprises of looped-shaped struts (FIG. 14A) and independent struts (FIG. 14B), respectively, in an expanded, deployed configuration and including a flexible printed circuit board (PCB) assembly thereon.

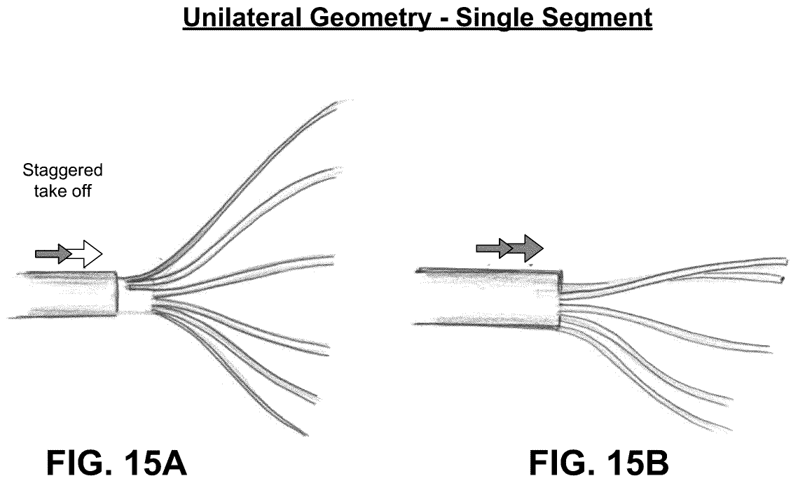

FIGS. 15A and 15B are side views of one embodiment of a framework of the single segment having a unilateral geometry in which the independent struts have a staggered take off section, resulting in transitioning of each independent strut between a retracted configuration and expanded, deployed configuration in a stepwise manner, such that specific struts contact locations at the target site in a sequential manner.

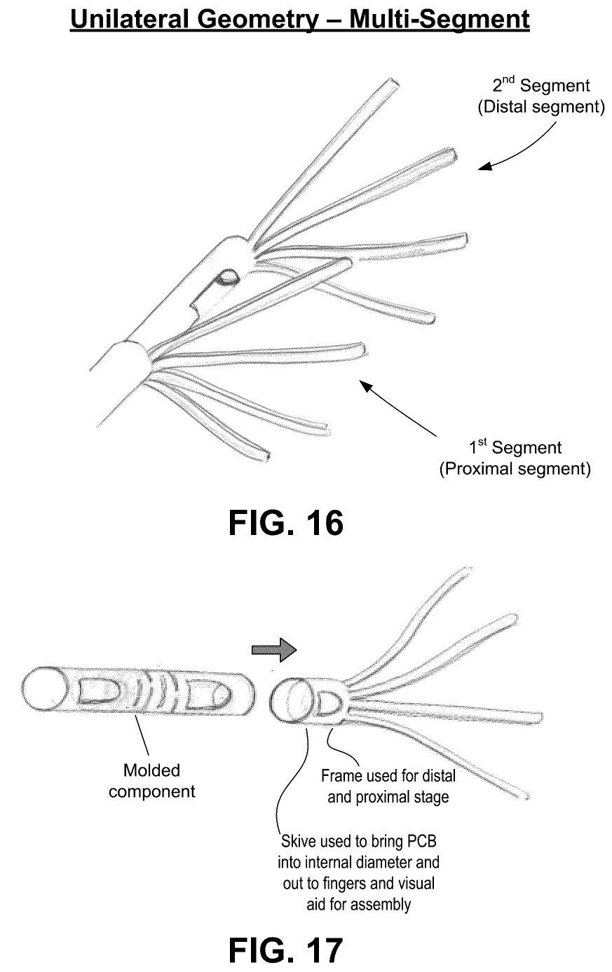

FIG. 16 is a perspective view of an embodiment of a multi-segment end effector composed of two segments that each have a unilateral geometry. FIG. 17 is a perspective view illustrating attachment of an interstage component to a given segment of the multi-segment end effector.

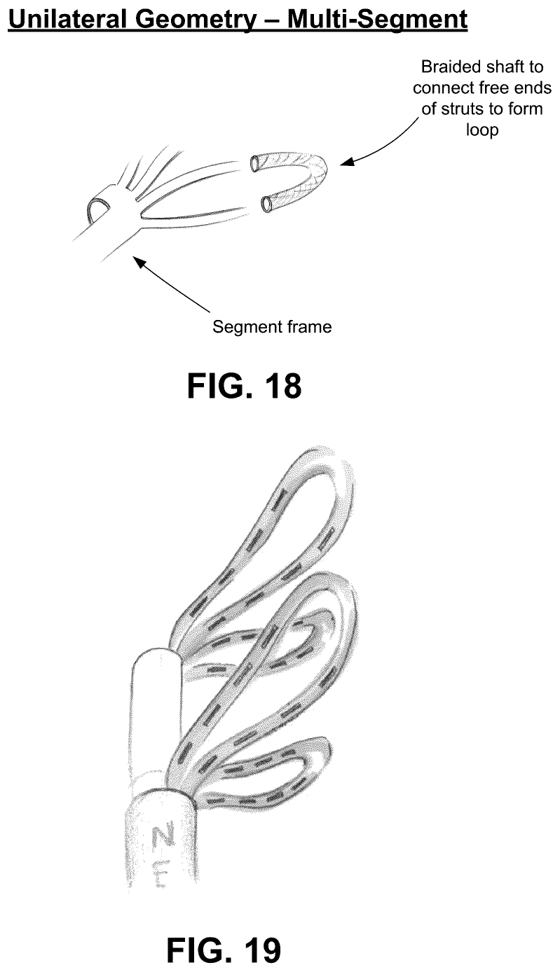

FIG. 18 is a perspective view illustrating joining of adjacent independent struts to one another via placement of a wire or braided tube over respective distal ends of the adjacent struts.

FIG. 19 is a perspective view of a multi-segment end effector having at least two segments each having a unilateral geometry and comprising looped-shaped struts in an expanded, deployed configuration and including a flexible printed circuit board (PCB) assembly thereon.

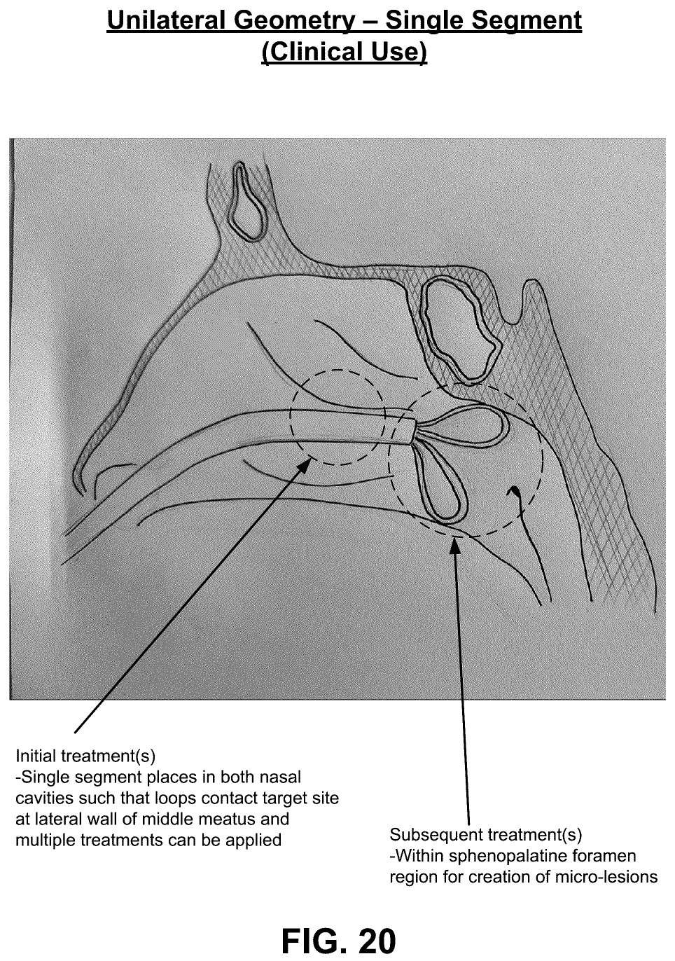

FIG. 20 is a cut-away side view illustrating one approach for delivering a single segment end effector having a unilateral geometry to a target site within a nasal region in accordance with embodiments of the present disclosure. Due to the unilateral design, the end effector can be placed in both nasal cavities so that the electrode array provided on the struts of the segment contact tissue sites at the lateral wall of the middle meatus in each cavity and energy can be delivered. The end effector can then be used for subsequent treatments in the sphenopalatine foramen (SPF) region so that multiple micro lesions can be created along the lateral wall.

As previously described, the handle includes multiple user-operated mechanisms, including at least a first mechanism 126 for deployment of the end effector from the collapsed/retracted configuration to the expanded deployed configuration and a second mechanism 128 for controlling of energy output by the end effector, notably energy delivery from one or more electrodes.

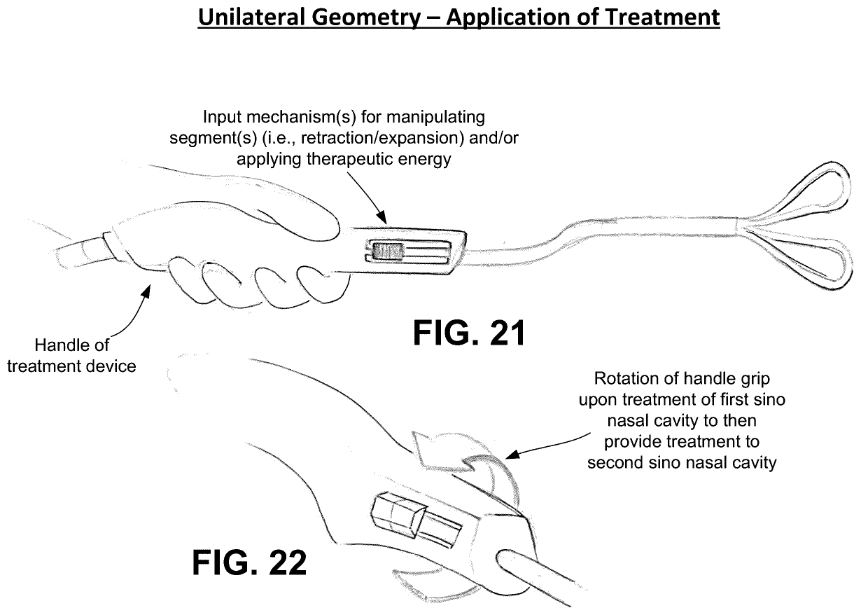

For at least the unilateral geometry design, a user must necessarily further manipulate the handle so as to cause the end effector to move to a complementary portion of a target site to receive treatment. For example, FIGS. 21 and 22 show user interaction with a handle of the treatment device of the present disclosure, including application of therapeutic treatment to target sites via a single segment having a unilateral geometry. For a segment having a unilateral geometry, the same struts provide therapeutic energy to both sino-nasal cavity target sites. For example, a user may first enter the left sino-nasal cavity and deploy the end effector along the middle turbinate for initial treatment of the lateral wall. Upon delivering energy thereto, the user need only rotate the handle 180 degrees about its axis to thereby correspondingly rotate the end effector such that the struts can oppose the lateral wall in the right sino-nasal cavity and then subsequently deliver energy thereto.

As previously described herein, the present invention further provides unique mechanisms for deploying the end effector (i.e., transitioning a given segment from between a retracted configuration and an expanded, deployed configuration).

In particular, in one embodiment, a treatment device includes a mechanical expansion assembly, which is generally in the form of a sphere or the like, that coupled to an input mechanism of a handle of the device via a pull rod or wire.

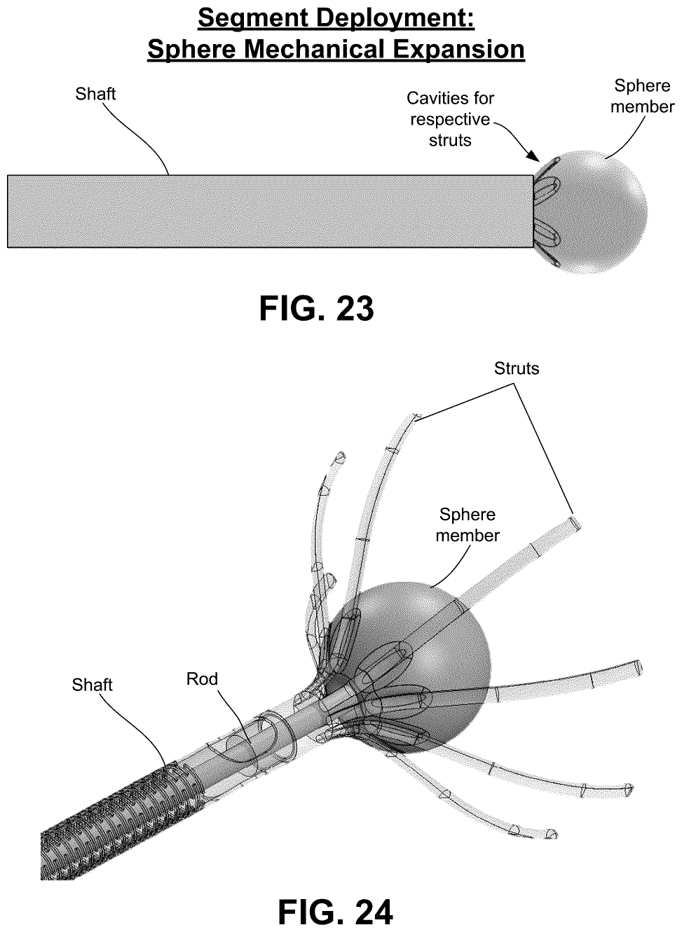

FIG. 23 is a side view of a polymer sphere mechanical expansion assembly in which the segment of the end effector is in a retracted configuration. FIG. 24 is a perspective view, partly in phantom, illustrating the polymer sphere mechanical expansion assembly and further showing the segment of the end effector in an expanded, deployed configuration. The sphere is generally positioned at a distal-most end of an end effector, specifically positioned so as to be drawn towards a center of a segment and thereby cause support elements or struts of the given segment to expand and transition to the deployed configuration upon user input with the pull rod or wire. More specifically, a user need only pull back on the pull rod or wire, which in turn draws the sphere towards the handle, thereby forcing the support elements or struts of the segment to extend outwardly and in a radial direction so as to expand.

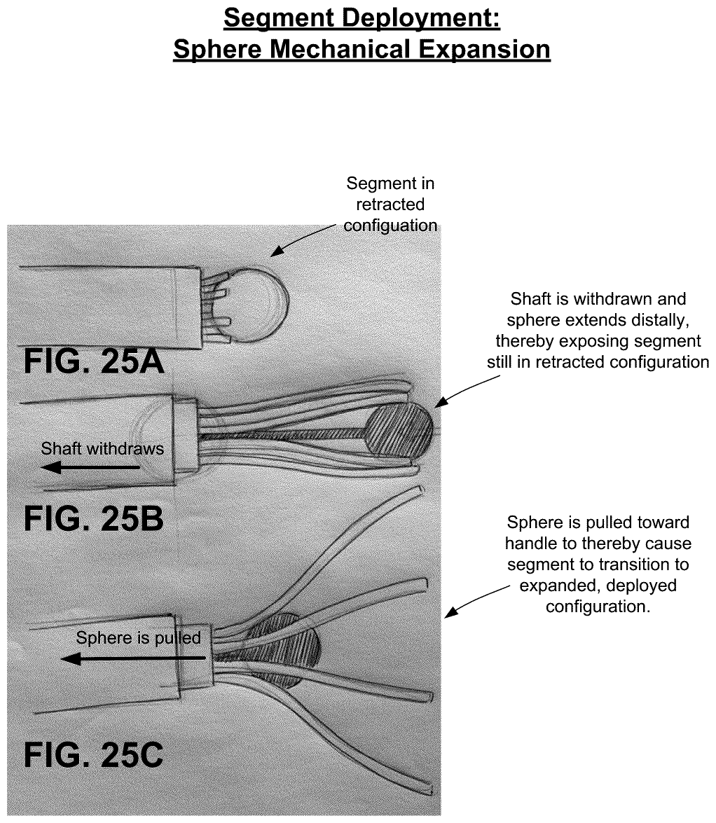

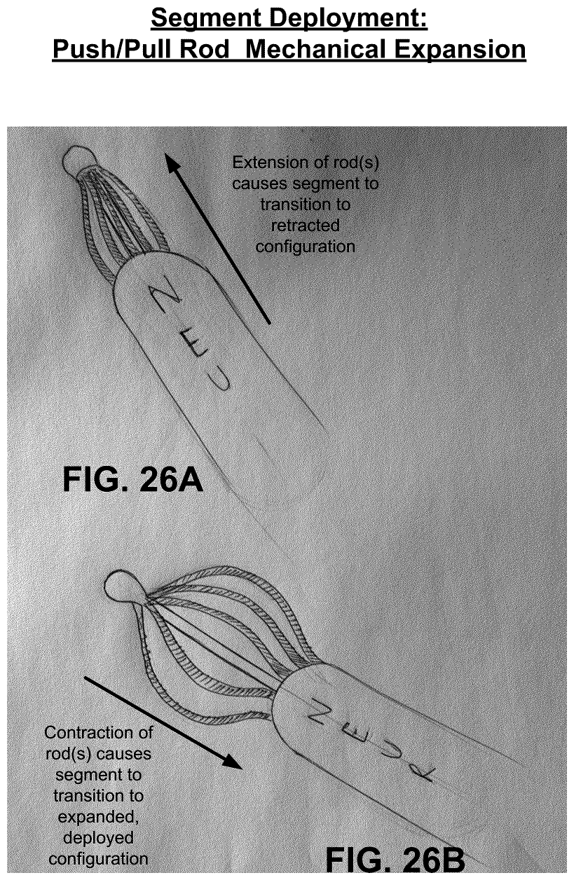

FIGS. 25A, 25B, and 25C illustrate transitioning of the segment of the end effector from a retracted configuration to an expanded, deployed configuration based on user manipulation of the polymer sphere mechanical expansion assembly.

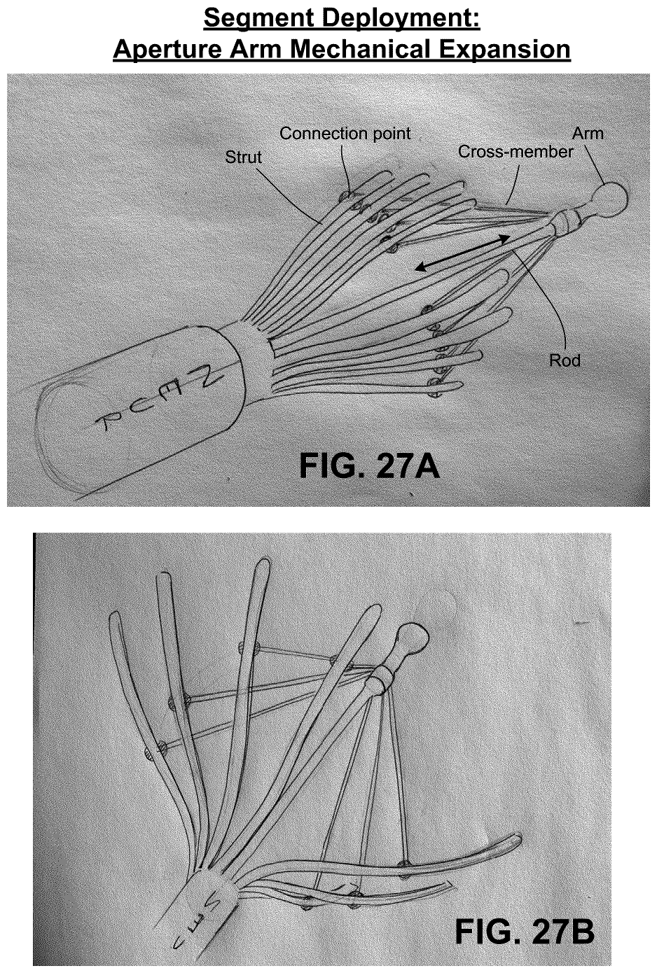

In another embodiment, the mechanical expansion assembly includes a push/pull rod assembly. FIGS. 26A and 26B are perspective views illustrating another embodiment of a mechanical expansion assembly that includes a push/pull rod assembly for transitioning a segment of the end effector between the retracted configuration (FIG. 26A) and the expanded, deployed configuration (FIG. 26B).

For example, in one embodiment, a segment may include multiple struts or support elements that are connected to one another at a distal-most end, such as a cap or the like. A push/pull rod may have a distal-most end coupled to the cap and a proximal-most end associated with an input mechanism of a handle of the device. Accordingly, a user need only manipulate the input mechanism to either cause the push/pull rod to be drawn towards the handle, thereby drawing the cap towards the handle and resulting in the segment expanding in a radial direction and transition to a deployed configuration or cause the push/pull rod to extend away from the handle, thereby pushing the cap away from the handle and resulting in the segment contracting and transitioning to a lower-profile, retracted configuration.

FIGS. 27A and 27B are perspective views illustrating another embodiment of a mechanical expansion assembly that includes a push/pull rod assembly for transitioning a segment of the end effector between the retracted configuration (FIG. 27A) and the expanded, deployed configuration (FIG. 27B). For example, the mechanical expansion assembly includes an aperture arm (in the form of a cap or the like) that is coupled to each of the struts or support elements of the segment via respective cross members. The arm is positioned at a distal-most end of a push/pull rod. Accordingly, a user need only manipulate an input mechanism on the handle to either cause the push/pull rod to be drawn towards the handle, thereby drawing the aperture arm towards the handle and resulting in the segment expanding in a radial direction and transition to a deployed configuration or cause the push/pull rod to extend away from the handle, thereby pushing the arm away from the handle and resulting in the segment contracting and transitioning to a lower-profile, retracted configuration. The various mechanical expansion assemblies improve contact between energy delivering elements of the end effector and the target sites within the nasal cavity, thereby improving the overall chances of achieving successful treatment.

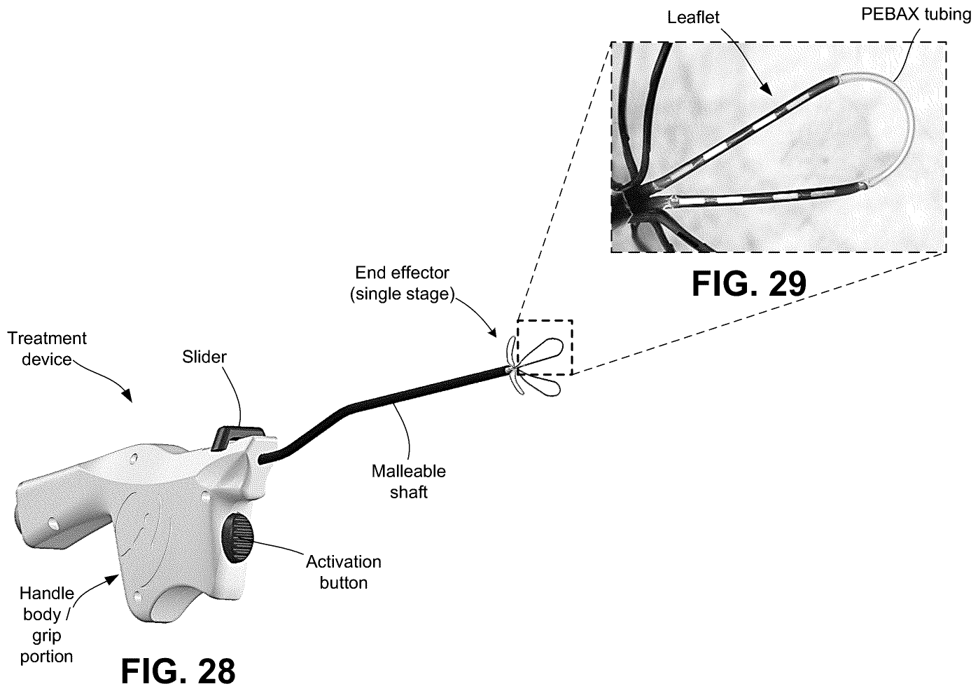

FIG. 28 is a perspective view of one embodiment of a handheld device for providing therapeutic treatment consistent with the present disclosure. As shown, the handheld treatment device includes an end effector transformable between a retracted configuration and an expanded deployed configuration, a shaft operably associated with the end effector, and a handle operably associated with the shaft. The end effector is shown as a single segment and is transformable between a retracted configuration, which includes a low-profile delivery state to facilitate intraluminal delivery of the end effector to a treatment site within the sino-nasal region, and a deployed configuration, which includes an expanded state (as shown in FIG. 28). The handle may include at least a first mechanism, in the form of a sliding input, for deployment of the end effector from the retracted configuration to the deployed configuration and a second mechanism, in the form of a button, for control of energy output from energy delivering elements of a given segment, specifically electrodes or other energy elements provided by a given segment.

For example, the slider mechanism is positioned so as to allow an operator (i.e., surgeon, clinician, medical professional, etc.) to easily interact with and manipulate the slider comfortably with one of their fingers or thumb depending on the preferred grip with which they are holding the device. The second mechanism may include button or trigger mechanism on the front end of the handle body, which facilitates user input so that selections on the console interface can be made and energy delivery can be initiated from the RF generator to the end effector. It should further be noted that the device may include a larger stock on handle body, which optimizes user interaction with the device and facilitates a more intuitive underhand grip. Furthermore, the shaft may be malleable and can be manipulated by user so as to deform it in multiple directions to thereby facilitate improved contact between the end effector and tissue sites in a given nasal cavity.

As previously described herein, the underlying design of the end effector is unique. For example, in the illustrated embodiment, the end effector includes a single segment having a bilateral geometry. In particular, the single segment includes two identical sides, including a first side formed of struts and a second side formed of struts. This bilateral geometry allows at least one of the two sides to conform to and accommodate an anatomical structure within the sino-nasal cavity when the segment in an expanded state. For example, when in the expanded state, the plurality of struts contact multiple locations along multiple portions of the anatomical structure and electrodes provided by the struts are configured to emit energy at a level sufficient to create multiple micro-lesions in tissue of the anatomical structure that interrupt neural signals to mucus producing and/or mucosal engorgement elements.

For example, the bilateral geometry refers to the fact that segments of one side of the end effector accommodate various anatomical locations along the lateral wall of one nasal cavity (i.e., left nasal cavity), while segments on the opposing side of the end effector accommodate locations on the lateral wall within the other nasal cavity (i.e., right nasal cavity). It should be noted that, in other embodiments, the end effector is multi-segmented in that it is composed of two or more segments, each having a bilateral geometry as previously described. Furthermore, in other embodiments, the end effector may include a single segment or multiple segments, each having a unilateral geometry.

The advantage of the single stage bilateral geometry is that it is simpler from a design and cost perspective and can be used to treat multiple locations in the middle meatus and posterior to the middle turbinate along the lateral wall of each nasal cavity.

FIG. 29 is an enlarged view of a portion of the single segment end effector of FIG. 28 illustrating a looped-shaped strut consistent with the present disclosure. As shown, the end effector is formed from a framework of loop-shaped struts. The support elements or struts are generally in the form of wires that have elastic properties. For example, in some embodiments, the struts may include a shape memory material, such as nitinol. The flexible support elements may further include a highly lubricious coating, which may allow for desirable electrical insulation properties as well as desirable low friction surface finish. Each of the struts is transformable between a retracted configuration and an expanded deployed configuration such that the struts are configured to position one or more electrodes provided thereon into contact with one or more target sites when in the deployed configuration.

It should be noted that the struts generally serve as an underlying framework upon which energy delivering elements (i.e., electrodes or the like) can be positioned. For example, as previously noted, the end effector of the present invention may further include one or more flexible printed circuit board (PCB) members operably associated therewith. Accordingly, any given segment of the end effector essentially serves as a framework upon which separate respective flexible PCB assemblies are attached, wherein energy delivering elements, such as electrodes, are provided via flexible PCB members.

In this manner, the present invention provides an end effector that is capable of highly conforming to anatomical variations within a sino-nasal cavity so that an operator can perform an accurate, minimally invasive, and localized application of energy to one or more target sites within the sino-nasal cavity of the patient to thereby treat a sino-nasal condition. In particular, unlike other surgical treatments for rhinitis, the devices of the invention are minimally invasive. Once delivered within the sino-nasal cavity, each segment of the end effector can expand to a specific shape and/or size corresponding to anatomical structures within the sino-nasal cavity and associated with the target sites. A plurality of flexible PCB members attached to a given segment of the end effector are able to correspondingly move and transition into the specific geometry of the given segment, such that, once deployed, the flexible PCBs contact and conform to a shape of the respective locations, including conforming to and complementing shapes of one or more anatomical structures at the respective locations.

In turn, the plurality of flexible PCB members become accurately positioned within the sino-nasal cavity to subsequently deliver, via one or more electrodes, precise and focused application of energy to targeted tissue at the one or more target sites, to disrupt multiple neural signals to, and/or result in local hypoxia of, mucus producing and/or mucosal engorgement elements, thereby reducing production of mucus and/or mucosal engorgement within a nose of the patient and reducing or eliminate one or more symptoms associated with at least one of rhinitis, congestion, and rhinorrhea.

The use of flexible PCBs members results in a greater amount of usable surface area than what is otherwise available with existing end effectors. In particular, the increase in surface area allows for a greater number of energy delivery elements to be introduced and utilized in a given procedure and further expands the possible number of patterns of such energy delivery elements. As a result, the contact surface increases substantially, thereby allowing for the end effector of the present invention to deliver treatment to certain areas within the sino-nasal cavity that may have been previously unreachable or untreatable with current treatment devices, or that previously required a surgeon to reposition a given device to reach such areas. Furthermore, the use of flexible PCB members reduces the overall complexity with regard to manufacturing the end effector of the present invention. In particular, any given flexible PCB member (including an overall PCB assembly, which includes multiple PCB members) is constructed separately from the end effector, which includes constructed the overall electrode design and placement on a given PCB member. Once a PCB assembly is complete, PCB members are then attached to respective portions of a given segment of the end effector as a separate manufacturing step, thereby reducing the complexity that is otherwise associated with placing electrodes directly on the end effector, which is a common practice. It should be noted, however, that, in some embodiments, a given flexible PCB member can be integrated into a molded segment during manufacture.

As shown, when in the expanded deployed configuration, the loop-shaped struts extend outward from a central axis to form an open-ended circumferential shape. In particular, the open-ended circumferential shape of the single segment shown in FIG. 28 generally resembles a blooming flower, wherein each looped strut may generally resemble a flower petal or leaflet. It should be noted that a given segment may include any number of individual struts and is not limited to the number shown. For example, as shown in FIGS. 28 and 29, the segment may include approximately four looped struts, such that there are approximately two leaflets or looped struts per side of the end effector (due to the bilateral geometry).

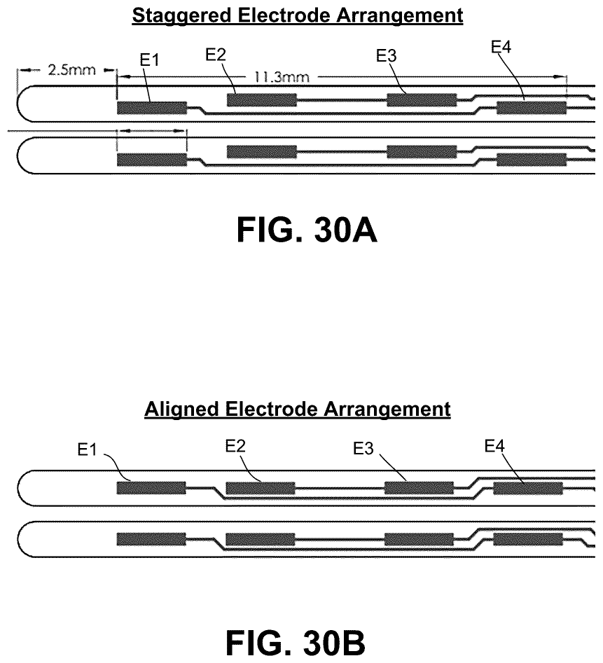

FIGS. 30A and 30B are plan views of exemplary electrode arrangements provided on loop-shaped struts. FIG. 30A shows a staggered electrode arrangement and FIG. 30B shows an aligned electrode arrangement. Energy is multiplexed to each leaflet through a single channel for each leaflet. As shown, there may be approximately four pairs of electrodes per leaflet. The aligned electrode arrangement is generally preferred.

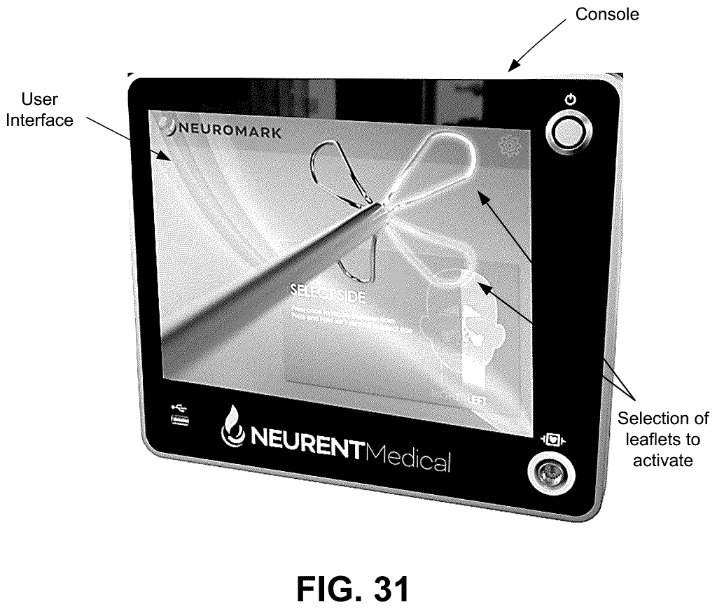

FIG. 31 is a perspective view of an exemplary user interface provided on a display either provided directly on the console unit or provided via a tablet or the like operably coupled to a console unit. As previously noted, feedback may be provided to an operator before, during, and/or after a treatment procedure. For example, information associated with the operation of the treatment device can be communicated to the operator via a graphical user interface (GUI) provided via a display on the console and/or a separate display communicatively coupled to the console such as a tablet or monitor, to thereby provide visual and/or audible alerts to the operator. The GUI may generally provide operational instructions for the procedure, such as directing the operator to select which sino-nasal cavity to treat, indicating when the device is primed and ready to perform treatment, input selections for the operator to choose, including, among other things, selection of specific leaflets to be activated during a given treatment, and further providing status of therapy during the procedure, including indicating when the operator should reposition the end effector to specific target locations, as well as when specific stages of treatment are complete (i.e., when treatment is complete at a first position/target location, treatment is complete at a second position/target location, etc.).

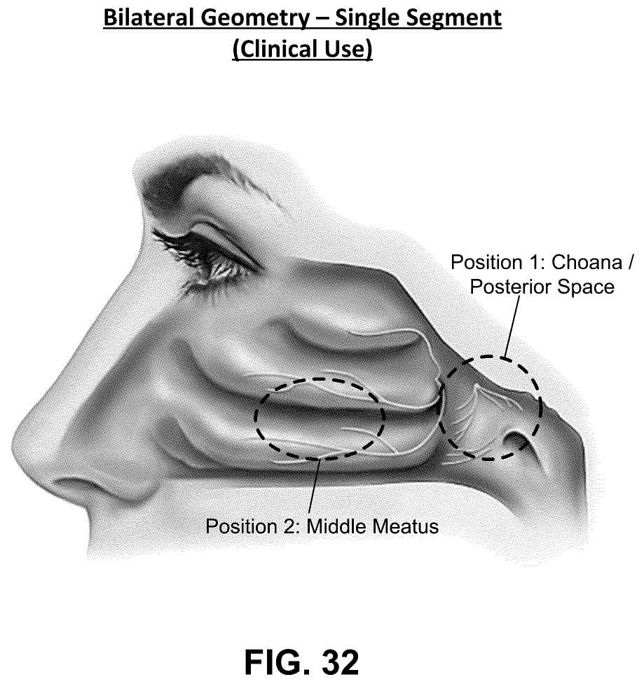

FIG. 32 is a side view, partly in section, illustrating the anatomy of a sino-nasal cavity specific locations within the sino-nasal cavity that a single segment end effector having a bilateral geometry is positioned for delivery of treatment energy in accordance with embodiments of the present disclosure. As shown, a first target site is at a posterior location within the sino-nasal cavity, specifically within the choana/posterior space, while a second target site is at an anterior location that is adjacent the middle meatus.

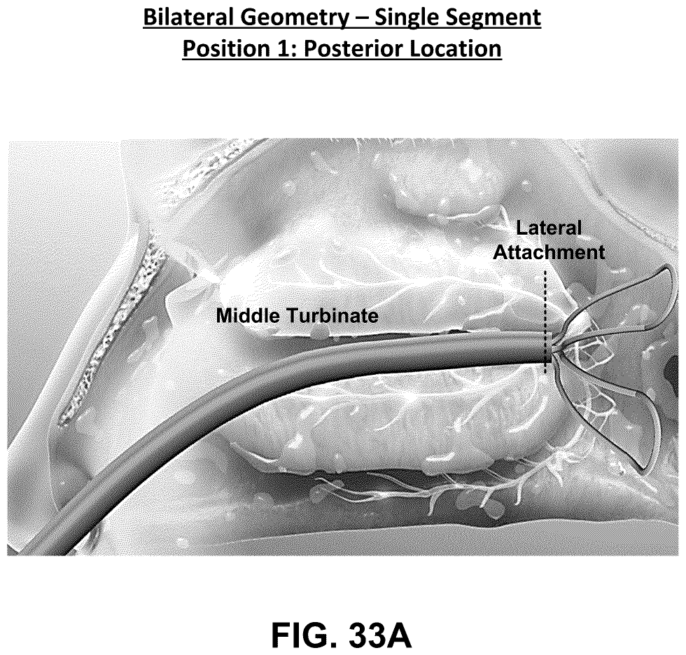

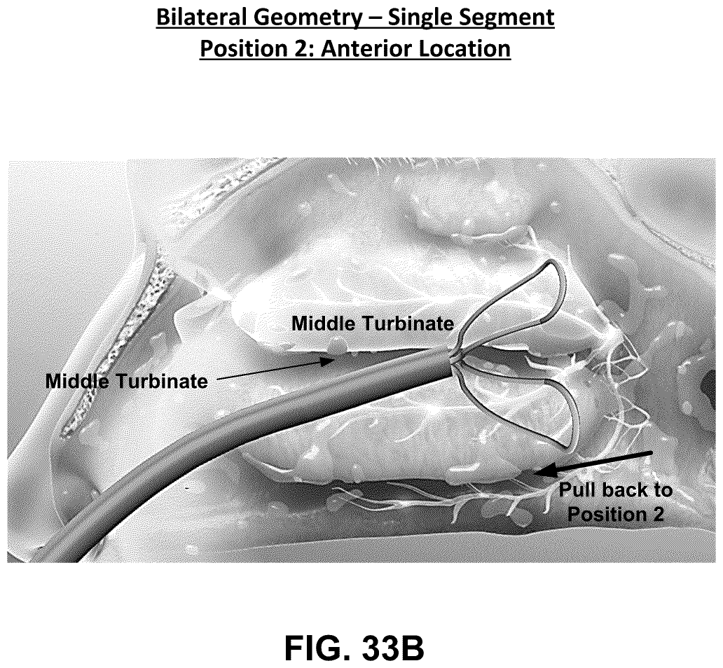

FIGS. 33A and 33B are side sectional views illustrating positioning of the single segment end effector at a first position (i.e., at the posterior location) and a second position (i.e., at the anterior location, respectively, within the sino-nasal cavity.

A treatment procedure may include the physician inserting and advances the distal end of the shaft into a first nasal cavity (maintaining the end effector in a retracted state) until the distal end of the shaft is at the first target site at the posterior location (i.e., in the choana, distal to the lateral attachment of the middle turbinate). The physician uses the slider mechanism on the handle to deploy the end effector so that two leaflets of the single stage end effector are deployed and in an expanded state and are apposing the lateral wall in the posterior location. Once the two leaflets are in apposition (i.e., in position 1), energy delivery to tissue can be initiated via the activation button. When energy delivery is complete, one of the two leaflets is available again for energy delivery. The end effector can then be repositioned to appose the lateral wall in the posterior space. Once apposition is regained, energy delivery can be initiated again via the activation button.

The physician can then pull the end effector (while in the expanded state) to the second target site at the anterior location, such that both leaflets are apposing the lateral wall of the middle meatus. Once both leaflets are in apposition (i.e. in position 2), energy delivery to tissue can be initiated via the activation button. After energy delivery, the device can be removed from the nasal cavity (i.e., the end effector can be transitioned to the retracted state and the shaft can be withdrawn from the sino-nasal cavity).

The physician can then perform the same steps for the second nasal cavity, in which the end effector is maintained in a retracted state, the distal end of the shaft is advanced within the second nasal cavity, and the same procedural steps employed in the first nasal cavity are followed.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

1. A device for treating a condition within a sino-nasal cavity of a patient, the device comprising:
an end effector comprising at least one retractable and expandable segment comprising a micro-electrode array arranged about a plurality of struts having a bilateral geometry and configured to conform to and accommodate an anatomical structure within the nasal cavity when the at least one segment is in an expanded state,
wherein the plurality of struts comprises:
a first set of struts extending outwardly in a first direction from a central axis of the at least one segment and configured to conform to and accommodate a first anatomical structure within a first nasal cavity; and
a second set of struts extending outwardly in a second direction from the central axis of the at least one segment and configured to conform to and accommodate a second anatomical structure within a second nasal cavity.

2. The device of claim 1, wherein the micro-electrode array is provided via one or more flexible printed circuit board (PCB) members positioned on one or more of the plurality of struts.

3. The device of claim 2, wherein the one or more flexible PCB members comprises a PCB substrate and one or more electrodes configured to deliver energy to tissue at the one or more target sites.

4. The device of claim 3, wherein each of the one or more flexible PCB members comprises one or more electrical communication paths positioned at least on or within the PCB substrate and selectively coupling the one or more electrodes to a corresponding one or more electrical contacts configured to electrically couple the one or more electrodes to a controller.

5. The device of claim 3, wherein the PCB substrate of the one or more flexible PCB members comprises a flexible material configured to correspondingly transition from a collapsed configuration to a deployed configuration upon movement of the segment to the expanded state.

6. The device of claim 5, wherein each of the plurality of struts includes at least a portion of one or more flexible PCB members fixedly coupled thereto.

7. The device of claim 1, wherein at least one of the plurality of struts is configured in a loop-like or leaflet-like shape when the first segment is in an expanded state.

8. The device of claim 1, wherein at least one of the plurality of struts has a distal-most end that is independent and separate from the other plurality of struts.

9. The device of claim 1, wherein each of the plurality of struts comprises a deformable material selected from the group consisting of a polymer and a shape memory material.

10. The device of claim 1, wherein the least one segment is a unitary single piece of material.

11. A device for treating a condition within a sino-nasal cavity of a patient, the device comprising:
an end effector comprising at least one retractable and expandable segment comprising a micro-electrode array arranged about a plurality of struts having a unilateral geometry and configured to conform to and accommodate an anatomical structure within the nasal cavity when the at least one segment is in an expanded state,
wherein the plurality of struts extend outwardly in a first direction from a central axis of the at least one segment and are configured to conform to and accommodate a first side of the anatomical structure.

12. The device of claim 11, wherein the micro-electrode array is provided via one or more flexible printed circuit board (PCB) members positioned on one or more of the plurality of struts.

13. The device of claim 12, wherein the one or more flexible PCB members comprises a PCB substrate and one or more electrodes configured to deliver energy to tissue at the one or more target sites.

14. The device of claim 13, wherein each of the one or more flexible PCB members comprises one or more electrical communication paths positioned at least on or within the PCB substrate and selectively coupling the one or more electrodes to a corresponding one or more electrical contacts configured to electrically couple the one or more electrodes to a controller.

15. The device of claim 13, wherein the PCB substrate of the one or more flexible PCB members comprises a flexible material configured to correspondingly transition from a collapsed configuration to a deployed configuration upon movement of the segment to the expanded state.

16. The device of claim 15, wherein each of the plurality of struts includes at least a portion of one or more flexible PCB members fixedly coupled thereto.

17. The device of claim 11, wherein at least one of the plurality of struts is configured in a loop-like or leaflet-like shape when the first segment is in an expanded state.

18. The device of claim 11, wherein at least one of the plurality of struts has a distal-most end that is independent and separate from the other plurality of struts.

19. The device of claim 11, wherein each of the plurality of struts comprises a deformable material selected from the group consisting of a polymer and a shape memory material.

20. The device of claim 11, wherein the least one segment is a unitary single piece of material.
